(19) United States
(12) Patent Application Publication
(10) Pub. No.: US 2021/0260201 A1
(43) Pub. Date: Aug. 26, 2021

(54) EXTRACELLULAR VESICLES FOR THE TREATMENT AND PREVENTION OF INFECTIONS AND OTHER DISEASES

(71) Applicant: PHYSIS BIOTECHNOLOGIES, LLC, Tucson, AZ (US)

(72) Inventors: Angela Chukly, Tucson, AZ (US); Emery Chukly, Tucson, AZ (US)

(73) Assignee: PHYSIS BIOTECHNOLOGIES, LLC, Tucson, AZ (US)

(21) Appl. No.: 17/172,293

(22) Filed: Feb. 10, 2021

Related U.S. Application Data
(60) Provisional application No. 62/979,722, filed on Feb. 21, 2020.
(60) Provisional application No. 63/016,509, filed on Apr. 28, 2020.

(51) Int. Cl. A61K 47/64; C07K 16/10

(52) U.S. Cl. CPC A61K 47/64; C07K 16/10

(57) ABSTRACT

In various embodiments, drug-loaded extracellular vesicles and methods of making the same are disclosed. In various embodiments, the EVs comprise natural exosomes or exomeres derived from cells that take part in the pathogenesis of a disease or a condition to which the drug-loaded EVs are targeted. In various embodiments, extracellular vesicles are surface modified, comprising bound biomolecules such as proteins or antibodies that can target ACE2 receptors. Surface modified EVs find use in the treatment and prevention of viral infections, such as COVID-19 caused by a SARS-CoV-2 infection in a human individual.

### CROSS-REFERENCE TO RELATED APPLICATIONS

This application claims priority to, and the benefit of, U.S. Provisional Patent Application Ser. No. 62/979,722 filed Feb. 21, 2020, entitled “Compositions Comprising Bioactive Loaded Extracellular Vesicles and Methods Thereof,” and U.S. Provisional Patent Application Ser. No. 63/016,509 filed Apr. 28, 2020, entitled “Surface Modified and Drug-Loaded Extracellular Vesicles for the Treatment and Prevention of Infections and Other Diseases.” These disclosures are incorporated herein by reference in their entireties for all purposes.

### FIELD

This disclosure generally relates to extracellular vesicles, and more specifically, to the use of extracellular vesicles as a vector for promoting human and non-human animal health and for the treatment of various diseases such as SARS-CoV-2 infection.

### BACKGROUND

Administration of bioactive substances to both humans and non-human animals (e.g., companion animals) can often be inefficient for a number of reasons. In the absence of targeted or controlled drug delivery, administration can typically result in the accumulation of the bioactive at any non-target site, leading to toxicity issues and poor physiological results. Controlled delivery of bioactive substances has improved recently, as well as targeted systems involving nanotechnology. Most of the research has involved surface manipulation of the bioactive-carrying nanoparticles with little attention afforded to other aspects of the targeted delivery system.

Many bioactive substances of interest in human and non-human animal health are unstable, poorly water soluble, too fat soluble, poorly intestinally absorbed, easily metabolized in the gut or liver, unable to cross the blood brain barrier, or simply unselective at the cellular level, such as between tumor or infected cells and normal cells.

Treatment of viral infections presents unique challenges. Currently, the ultimate way to overcome spread of a virus in the human or animal population is to use a vaccine. Vaccines work by stimulating the host organism's immune system to produce antibodies exactly like it would if the host was exposed to the disease. After getting vaccinated, the host organism develops an immunity to that disease or pathogen without having to get the actual disease. The mechanism of creating a vaccine requires generating an antigen from the pathogen that will induce an immune response. However, a typical virus can mutate over time such that the antigen that was used to make the vaccine no longer recognizes the mutated virus, resulting in the vaccine losing its efficacy (i.e., in properly activating the adaptive immune system to recognize, activate the immune system, and attack the virus) Due to the viruses' ability to mutate, it is necessary to constantly generate new vaccines, which is costly, inefficient, and can result in delays before treatment is available, which during that time, a pandemic outbreak situation may occur. Presently the world is in such a pandemic with the onset and global spread of SARS-CoV-2, which is expectedly mutating into several global variants, presenting an on-going vaccine challenge.

In view of these and other shortcomings in healthcare, new drugs, pharmaceutical compositions, and drug treatment therapies are still needed for promoting human and non-human animal health and to combat global pandemics.

### SUMMARY

In various embodiments, new compositions comprising extracellular vesicles are described.

In various embodiments, encapsulation of bioactive substances within vesicles may increase the solubility and physiochemical stability of bioactive substances. In various embodiments, compositions comprising bioactive-loaded vesicles may provide rapid and targeted delivery of high concentrations of the bioactive when administered to a human or non-human animal.

In various embodiments, methods and compositions comprising extracellular vesicles may provide a vector for targeted delivery of bioactive substances in the treatment in vivo of various diseases in humans and non-human animals.

In various embodiments, methods and compositions comprising extracellular vesicles loaded ex vivo with one or more phytocannabinoids may provide a vector for phytocannabinoid treatment in vivo of various diseases.

In various embodiments, encapsulation of phytocannabinoids in vesicles such as exosomes, and exploitation of these delivery systems, may provide excellent solutions that overcome some of the major problems in the advancement of Cannabis based therapies. In various embodiments, phytocannabinoids are packaged in exomeres 50 nm diameter particles).

In various embodiments, surface modified extracellular vesicles isolated from mesenchymal stem cells are used for the treatment and prevention of acute SARS-CoV-2/COV19 infections.

In various embodiments, compositions comprising extracellular vesicles, or surface modified extracellular vesicles, optionally loaded ex vivo with one or more bioactive substances, may be in the form of oils, crèmes, powders, nasal sprays, oral mucosal sprays, and liquids suitable for inhalers and nebulizers.

In various embodiments, compositions comprising extracellular vesicles find use in personal care, e.g., age spot removers, dark spot correctors, anti-aging creams, antifungal treatment (nails, skin), and the like.

In various embodiments, a composition for inhalation through an inhaler or nebulizer comprises surface modified extracellular vesicles isolated from mesenchymal stem cells are used for the treatment and prevention of acute SARS-CoV-2/COV19 infections.

In various embodiments, a composition is disclosed comprising extracellular vesicles, the extracellular vesicles comprising at least one bioactive substance loaded therein.

In various embodiments, the bioactive substance comprises at least one of an antibody, antibiotic, antiviral, antifungal, antitumor, a protein, a nucleic acid, an oligonucleotide, or a chemotherapeutic.

In various embodiments, the bioactive substance comprises at least one of THC or CBD.

In various embodiments, the extracellular vesicles comprise at least one of exosomes or exomeres.

In various embodiments, the composition is a liquid, oil, cream, or powder.

In various embodiments, a method of treating a disease in a human or non-human animal comprises administering to the human or non-human animal in need thereof a therapeutically effective amount of a composition comprising extracellular vesicles loaded with at least one bioactive substance.

In various embodiments, the disease to be treated comprises a SARS-CoV-2 infection, cancer, mental illness, a neurodegenerative disease, an autoimmune disease, or epilepsy.

In various embodiments, the bioactive substance comprises at least one of an antibody, antibiotic, antiviral, antifungal, antitumor, a protein, a nucleic acid, an oligonucleotide, a chemotherapeutic or a phytochemical.

In various embodiments, the bioactive substance comprises at least one of a combination of the two monoclonal antibodies casirivimab and imdevimab, or the antiviral active remdesivir.

In various embodiments, the extracellular vesicles comprise at least one of exosomes or exomeres.

In various embodiments, a method of managing pain in a human or non-human animal comprises administering to the human or non-human animal in need thereof a therapeutically effective amount of a composition comprising extracellular vesicles loaded with at least one phytocannabinoid or endocannabinoid.

In various embodiments, the extracellular vesicles comprise at least one of exosomes or exomeres.

In various embodiments, a method of treating a SARS-CoV-2 infection in a human patient comprises administering to the patient in need thereof a composition comprising extracellular vesicles containing at least one of an antibody or antiviral effective against SARS-CoV-2 virus encapsulated therein.

In various embodiments, the extracellular vesicles comprise mesenchymal stem cell derived extracellular vesicles further comprising a biomolecule attached thereon.

In various embodiments, the biomolecule comprises an ACE2 receptor antibody or a polypeptide capable of hybridizing to a portion of an ACE2 receptor.

In various embodiments, the biomolecule is selected from the group consisting of nucleotides, nucleosides, oligonucleotides, DNA, RNA, hybridization probes, amino acids, polypeptides, proteins and fragments thereof, and antibodies and fragments thereof

In various embodiments, the extracellular vesicles contain a mixture of casirivimab and imdevimab monoclonal antibodies encapsulated therein.

In various embodiments, the extracellular vesicles contain remdesivir encapsulated therein.

In various embodiments, a method of manufacturing drug-loaded exosomes or exomeres comprises culturing parental cells capable of naturally producing exosomes or exomeres; isolating the exosomes or exomeres; purifying the exosomes of exomeres; and incubating the purified exosomes or exomeres in a buffer comprising the drug for a time sufficient to load the exosomes or exomeres ex vivo with the drug, wherein the drug is at least one of an antibody, antibiotic, antiviral, antifungal, antitumor, a protein, a nucleic acid, an oligonucleotide, or a chemotherapeutic.

In various embodiments, the drug comprises at least one of a combination of casirivimab and imdevimab, or remdesivir.

### BRIEF DESCRIPTION OF THE DRAWING FIGURES

FIG. 1 is a flowchart outlining an exemplary method for producing compositions comprising bioactive loaded extracellular vesicles, in accordance with various embodiments;

FIG. 2 illustrates a flowchart showing an overall mechanism for development of MSC derived EVs for therapeutic use; and

FIGS. 3A and 3B illustrate a flowchart of steps for isolating and identifying an extracellular vesicle subset usable for a particular desired outcome.

### DETAILED DESCRIPTION

The detailed description of exemplary embodiments refers to the accompanying drawings, which show exemplary embodiments by way of illustration and best mode. While these exemplary embodiments are described in enough detail to enable those skilled in the art to practice the invention, other embodiments may be realized, and logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Compositions comprising extracellular vesicles are described. In various embodiments, the extracellular vesicles further comprise a bioactive or a combination of bioactive substances loaded therein. In various embodiments, extracellular vesicles are loaded ex vivo with one or more bioactive substances. The pre-loaded vesicles are then formulated into compositions usable to administer the one or more bioactive substances to a human or non-human animal in need thereof.

Extracellular vesicles as a whole encode a universal life language, and rigorous characterization, isolation, identification and analysis of extracellular vesicle subsets from pristine sources combined with current technology can be applied to unlock our understanding of this language. By generating a substantial database of extracellular vesicle subsets for a variety of applications (biomarkers, cancer, liquid biopsy, pathogens defense etc.), and using computational methodologies to analyze the “omics” data, it is possible the extracellular vesicle industry will evolve into a whole new drug platform that can be applied in a way to treat any organism independent of source in treatment for disease, achieving homeostasis, or biohacking a particular pathogen to prevent its ability to invade host cells (prevention).

In various embodiments, compositions herein comprises extracellular vesicles further comprising drug actives encapsulated therein, such as antibiotics, antibodies, proteins, nucleic acids, antibacterial agents, antifungal agents, antiviral agents, etc. In various examples, the drug active loaded in extracellular vesicles comprises the REGN-COV-2 antibody cocktail (from Regeneron®), comprising two noncompeting, neutralizing human

IgG1 antibodies that target the receptor-binding domain of the SARS-CoV-2 spike protein. This, and other drug actives are loaded ex vivo into the vesicles, wherein the drug actives are present in high concentration, and are more stable and more bioavailable than in conventional compositions absent extracellular vesicles.

In various embodiments, compositions herein comprise extracellular vesicles further comprising phytocannabinoids. The phytocannabinoids are loaded ex vivo into the vesicles, wherein the phytocannabinoids are present in high concentration, and are more stable and more bioavailable than in conventional compositions absent extracellular vesicles. In various embodiments, the compositions herein are in the physical form of oils, crèmes, powders, nasal sprays, oral mucosal sprays, and liquids suitable for nebulization.

### Definitions

As used herein, the term “extracellular vesicles,” or “EV” for short (“EVs” for plural), refers to cellular-derived lipid encased particles secreted by cells of multicellular organisms into external environments to achieve cellular homeostasis and/or for cell to cell communication. Further, the term is meant to include the entire family of extracellular vesicles, including the recently discovered exomeres (extracellular nanoparticles having no apparent biological function), along with engineered extracellular vesicles and mimetic nanovesicles. Natural EVs can be classified into three main types. Namely, apoptotic bodies (500-2000 nm in size), microvesicles (50-1000 nm in size), and exosomes (30-200 nm in size). Besides these size differentiations, the types of vesicles are also differentiated by biogenesis, release pathways, content and function. Exosomes formed by an endosomal route are typically 30-150 nm in diameter. There is no apparent limit as to the type or source of EVs for loading with bioactive substances and formulation into compositions in accordance with the present disclosure. EVs for use herein include, but are not limited to, EVs obtained from plasma, urine, semen, saliva, bronchial fluid, cerebral spinal fluid, breast milk, serum, amniotic fluid, synovial fluid, tears, lymph, bile, and gastric acid. In various embodiments, EV's are obtained from stem cells, for example, from mesenchymal stem cells (MSCs). In various embodiments, MSCs for use herein, as a source of EV's, may be derived from Wharton's jelly tissues (thus, Wharton's jelly-derived mesenchymal stem cells, or WJ-MSCs). Further, any method of isolation presently known or developed in the future may be used. Current methods for isolating EVs include ultracentrifugation, density gradient methods, size exclusion methods, immunoaffinity capture, and precipitation. For a comprehensive review, see L. M. Doyle, et al., “Overview of Extracellular Vesicles, Their Origin, Composition, Purpose, and Methods for Exosome Isolation and Analysis,” Cells, 8, 727 (2019).

As used herein, the terms “pharmaceutical active,” “drug active,” or more generally, “bioactive,” (used interchangeably herein), refer to any naturally occurring, semisynthetic or synthetic substance that elicits any sort of pharmacological effect in a human or non-human animal, including medicinal effects, homeopathic effects, prophylactic effects, sensory effects, placebo effects, and psychological effects. In other words, any naturally occurring, semisynthetic or synthetic substance that might be mentioned herein can be classifiable as either an active, that is, having any sort of interaction with the organs and systems in a human or non-human animal, or an inert, that is, something entirely unrecognizable by the human or non-human animal. An example of the former is lavender oil, which at least has a sensory effect and possibly a homeopathic effect. An example of the later is PFOS—perfluorooctane sulfonate, which is believed to be entirely inert to a human or non-human animal. Bioactive substances of interest herein include, but are by no means limited to, phytocannabinoids, endocannabinoids, any plant, tree, bark, root, tuber, fruit, nut or flower extract, bacteria, fungi, algae and their associated intracellular constituents, extracts, metabolites and derivatives thereof, such as saccharides and polysaccharides, and any naturally occurring small molecule (i.e., less than about 900 Dalton), such as terpenes, sesquiterpenes, alkaloids, glycerides, glycosides, lipids, flavonoids, peptides, oligonucleotides, alcohols, aldehydes, carboxylic acids, esters, ketones, and the like. In various embodiments, any single active or combination of actives may be loaded into a vesicle for administration to a human or non-human animal via a variety of compositions and delivery forms.

A bioactive substance for use herein may comprise a drug active recognizable by one skilled in the art of medicinal chemistry. Such bioactive substances for use herein include, but are not limited to, antibodies and antibody cocktails, nucleic acids, proteins, oligonucleotides including messenger-RNA (mRNA), antidiabetic agents, glucose elevating agents, thyroid hormones, thyroid drugs, parathyroid drugs, vitamins, antihyperlipidemic agents, cardiac drugs, respiratory drugs, nasal decongestants, gastrointestinal drugs, amphetamines, anorexiants, antirheumatic agents, anti-gout agents, migraine drugs, sedatives, hypnotics, antianxiety drugs, anticonvulsants, antidepressants, antipsychotic agents, psychotherapeutic drugs, antimicrobials, antifungals, sulfonamides, antimalaria drugs, antituberculotic drugs, amebicides, antiviral agents, anti-infectives, leprostatics, antihelmintics, antihistamines, antimetabolites, anticholinergics, steroidal anti-inflammatories, anesthetics, antiplatelet drugs, NSAIDs, ace inhibitors, calcium channel blockers, alpha-blockers, muscle relaxers, antihypertensives, vasodilators, diuretics, antiemetics, sex hormones, pituitary hormones, analgesics, uterine hormones, and adrenal steroid inhibitors.

As used herein, a “biomolecule” takes on its ordinary and broad meaning of naturally occurring, or synthetic macromolecules that are associated with a biochemical purpose in an organism. Biomolecules herein may be attached to the outer surface of extracellular vesicles, and such biomolecules may include nucleotides, nucleosides, oligonucleotides, DNA, RNA, hybridization probes, amino acids, polypeptides, proteins and fragments thereof, and antibodies and fragments thereof.

As used herein, the term “phytocannabinoid” refers to the general class of cannabinoids naturally produced in the Cannabis plant. In various embodiments, the terms phytocannabinoid and cannabinoid are used interchangeably, although the strict definition of phytocannabinoid might not include semisynthetic cannabinoids. Some cannabinoids, such as tetrahydrocannabinol (THC), can be obtained by laboratory decarboxylation of tetrahydrocannabinolic acid (THC-acid) (i.e., semisynthetic). But since THC is also found naturally occurring in Cannabis, the substance is also a phytocannabinoid. There are believed to be at least 113 phytocannabinoid species present in Cannabis.

As used herein, the term “endocannabinoid” refers to substances made by the human or non-human animal that activate cannabinoid receptors. Some of these substances, such as the endocannabinoid ligands, are also found in plants other than Cannabis. These substances are believed to act as intercellular lipid messengers in a cascade of events that activate cannabinoid receptors on nearby cells. Endocannabinoids of interest herein include, but are not limited to, anandamide (AED), 2-arachidonoylglycerol (2-AG), 2-arachidonoyl glyceryl ether, N-arachidonoyl dopamine, virodhamine (OAE), and lysophosphatidylinositol (LPI).

As used herein, the term “cannabinoid” broadly refers to those compounds having cannabinoid receptor type-1 or receptor type-2 activity, either found naturally occurring in various Cannabis plant species or other terrestrial plants, or obtained by synthetic organic transformations starting from naturally occurring substances. Cannabinoids of interest herein include, but are not limited to, tetrahydrocannabinol (THC), tetrahydrocannabinolic acid (THC-acid), cannabidiol (CBD), cannabidiolic acid (CBD-acid), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE) and cannabicitran (CBT). As evident from this list, some of the cannabinoids of interest herein are characterizable as carboxylic acids (e.g., THC-acid), and in various embodiments, it may be desirable to convert a particular cannabinoid carboxylic acid into its corresponding decarboxylated cannabinoid prior to formulation in composition.

As used herein, the term “composition” takes on the ordinary meaning in formulation chemistry as a combination of ingredients. In various embodiments, a composition is designed to adopt a particular physical form, or at least be amenable to a physical change into a desired physical form, which may be a dosage form for a treatment. Typically, a composition is made homogeneous by mixing or blending, although not all compositions are colorless and transparent. Compositions comprising an emulsion, dispersion or suspension may be homogeneous because the droplets or particles are evenly spread in a carrier. So, for example, a composition herein may be in the form of a thin liquid (having a viscosity at or near that of water), a viscous liquid (having a liquid of viscosity greater than water), a paste, a crème, a jelly, a gel, or a powder. An ingredient listing for a composition herein is generally shown “as added,” meaning that the ingredient list might not show that a chemical reaction takes place between two or more ingredients once the ingredients are mixed. One skilled in the art of formulation chemistry would recognize if ingredients might react in a mixture, such as neutralization (acid/base ingredients), mixed micelle formation (mixed surfactants), hydrolysis, and so forth. In various embodiments, a composition herein comprises a bioactive loaded vesicle, such as a drug loaded exosome.

As used herein, the term “dosage form” takes on its ordinary meaning in the pharmaceutical arts as the physical form of a composition designed for a particular administration route. For example, dosage forms include, but are not limited to, injectables, infusible liquids, nasal sprays, inhalable liquids, liquids for nebulization, nasal gels, topicals such as transdermal creams, ointments and patches, loose powders, tablets, capsules, lozenges, syrups, vapors, and so forth. In various embodiments, a dosage form may be packaged in a dosage-delivery system amenable to the dosage form. For example, an inhalable liquid dosage form may be packaged in an inhaler. A liquid for a nebulizer may be in a cartridge that is attachable to a nebulizer.

As used herein, the term “subject” or the phrase “a subject in need thereof” refers to any human or non-human animal requiring or desirous of a pharmacological change. For example, a subject in need thereof may be a human patient clinically diagnosed with a disease, such as epilepsy, and is thus in need of, or is at least desirous of receiving some sort of treatment. Also, a subject in need thereof may be a human desirous of a mood change. Or a subject in need thereof may be a canine animal having complications from arthritis. Most importantly, a subject in need thereof can be any human or non-human animal in perfect health, but desirous of maintaining good health. In other words, the subject in need thereof may be desirous of a prophylactic regimen, like taking daily vitamins.

As used herein, the term “treatment” of a subject (e.g., a mammal, such as a human) or a cell, is any type of intervention used in an attempt to alter the natural course of the subject or cell. Treatment includes, but is not limited to, administration of a vesicle-based composition in accordance with the present disclosure, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

As used herein, the term “therapeutically effective amount” refers to a minimum dosage of a composition in accordance with the present disclosure that provides a desired effect. Therefore, a therapeutically effective amount varies by subject, dosage form/concentration, and results desired. For example, a therapeutically effective amount of an injectable composition disclosed herein to treat cancer, or a therapeutically effective amount of an inhalable liquid to treat a respiratory viral infection, might be on the order of micrograms or milligrams per day. In other examples, a therapeutically effective amount of a nasal spray composition disclosed herein to heighten mood and reduce depression, might be on the order of 3 sprays per day per nostril, with each spray about 0.1 mL.

As used herein, the term “prophylactically effective amount” refers to a minimum dosage of a composition in accordance with the present disclosure that provides maintenance of a desired level of health, such as preventing the onset of a viral or other infection. Therefore, a prophylactically effective amount varies by subject, (particularly age, gender, and current health habits and ongoing issues), dosage form/concentration, and results desired. For example, a prophylactically effective amount of a tablet composition disclosed herein to promote general health in an elderly male in prostate cancer remission may be 3 tablets per day, with each tablet 500 milligrams. In other examples, a prophylactically effective amount of a nasal spray or inhaler composition disclosed herein for a 25 year old to maintain general health and reduce the likelihood of contracting a seasonal flu or emerging pathogenic virus might be on the order of 1 spray per day per nostril, or one inhaler delivery, with each spray or inhaled dosage about 0.1 mL.

As used herein, the term “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or more vesicle-based compositions in accordance with the present disclosure to produce or cause a greater physiological response (i.e., downstream effects) in a cell or in a subject relative to the response caused by either no vesicle-based composition or a control compound. Relevant physiological or cellular responses (in vivo or in vitro) upon administration of vesicle-based compositions will be apparent to persons skilled in the art. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7. 1.8), the amount produced by no vesicle-based composition (the absence of a bioactive agent) or a control compound. The term “reduce” or “inhibit” may relate generally to the ability of one or more vesicle-based compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease, illness or condition, such as cancer, inflammation or pain. A “decrease” in a response may be “statistically significant” as compared to the response produced by no vesicle-based composition or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

As used herein, the term “semisynthetic” refers to a bioactive substance that is obtained by one or more reactions in organic chemistry, beginning with a naturally occurring substance. In other words, a naturally occurring substance may need to undergo one or more synthetic steps to be ultimately useful.

As used herein, the term “synthetic” refers to a bioactive substance that is made entirely by organic synthesis, such as in a linear synthesis or convergent synthesis, possibly involving asymmetric synthesis to obtain specific enantiomers.

As used herein, the term “decarboxylation” refers to a reaction in organic chemistry whereby a carboxyl group is removed from a starting molecule, thus liberating CO₂. The reaction may be generalized by the reaction equation: R—CO₂H→R—H+CO₂, often referred to as hydro-decarboxylation because of the resulting hydrocarbon R—H. A well-known decarboxylation that fits this equation, which is of interest herein, is the decarboxylation of Δ⁹-tetrahydrocannabinolic acid (THC-acid) to Δ⁹-tetrahydrocannabinol (THC). In various embodiments, a cannabinolic acid, such as Δ⁹-tetrahydrocannabinolic acid (THC-acid) might be decarboxylated to the corresponding cannabinoid prior to incorporation inside a vesicle such as an exosome.

### General Embodiments

### I. Drug-Loaded Extracellular Vesicles

In various embodiments, compositions comprising extracellular vesicles are described, wherein the vesicles are loaded with a bioactive or a combination of bioactive substances. In various embodiments herein, EVs may be loaded ex vivo with a bioactive or a combination of bioactive substances. In various embodiments herein, EVs may comprise exosomes or exomeres, such as depending on the bioactive substances to be encapsulated and/or the disease state, and/or the preventative or treatment need.

### Drug Actives

Drug-loaded EVs may comprise any pharmaceutically bioactive substance, such as an antibody, antibiotic, antiviral, antifungal, antitumor, a protein, a nucleic acid, an oligonucleotide, or a chemotherapeutic. In various embodiments, the drug-loaded EV comprises a bioactive effective in treating a SARS-CoV-2 infection in a human patient in need thereof. In various embodiments, the active effective in treating a SARS-CoV-2 infection comprises REGN-COV2 antibody cocktail (combination of the two monoclonal antibodies casirivimab and imdevimab), or remdesivir (an antiviral).

### Cannabinoids

There is a growing body of evidence to suggest that cannabinoids are beneficial for a range of clinical conditions, including pain, inflammation, epilepsy, sleep disorders, the symptoms of multiple sclerosis, anorexia, schizophrenia, and other conditions. Cannabis sativa is a dioic plant that belongs to the Cannabaceae family (Magnoliopsida Urticales). Knowledge of the medical and psychoactive properties of Cannabis dates back to 4000 B.C. While more than 100 different cannabinoids can be isolated from C. Sativa, the primary psychoactive compound is Δ⁹-tetrahydrocannabinol (THC). Other pharmacologically important analogues are cannabidiol (CBD), cannabinols, cannabinoid acids, cannabigerols, and cannabivarins. Although phytocannabinoids in general have certain similar chemical structural features, they can elicit different pharmacological actions. The identification of THC paved the way for the discovery in 1988 of cannabinoids receptor type 1 (CB1), and later, of cannabinoid receptor type 2 (CB2). CB1 and CB2 belong to a family of seven transmembrane Guanosine Binding Protein Coupled receptors and are widely expressed and distinguished by their specific functions, localization and signaling mechanisms. The psychotropic effects of Cannabis are principally mediated by CB1, which is widely distributed throughout the brain, but mainly in the frontal cortex, basal ganglia, and cerebellum. CB1 is also present in several tissues and organs, including adipose tissue, the gastrointestinal tract, the spinal cord, the adrenal and thyroid glands, liver, reproductive organs and immune cells. CB2 is principally expressed in immune cells, but can also be found in other cell types, including chondrocytes, osteocytes, and fibroblasts, as well as evidence for their presence of fibroblast-like synoviocytes, makes CB1 particularly interesting in the study of rheumatic diseases.⁽¹⁾

However, cannabinoids are highly lipophilic molecules (logP=6-7) with very low aqueous solubility (2-10 μg/mL). Cannabinoids are also susceptible to degradation, especially in solution, via the action of light and temperature as well as via auto-oxidation.⁽¹⁾ Thus, packaging a cannabinoid in EVs can mitigate these shortcomings. Formulation can thus play a crucial role in increasing the solubility of physiochemical stability of the drugs. Encapsulation of phytocannabinoids with EVs (e.g., primarily exosomes) and exploiting these delivery systems may provide an excellent solution to overcome some of the major problems in the advancement of Cannabis based therapies. Most notably, phytocannabinoids and their metabolites primarily travel within EVs (in particular, exomeres), and isolation of a particular subset of EVs would enrich the therapeutic efficacy of phytocannabinoids.

### Extracellular Vesicles

Regardless of type, EVs contain both internal cargo such as cytoplasmic proteins, nucleic acids, and metabolites, as well as membranous payloads such as receptors, phospholipids, and adhesion molecules. The different types of EVs differ in their properties as well as their biogenesis. In addition, exosomes, the more extensively studied of all EV subtypes, can cross the blood brain barrier (BBB), and can be detected peripherally, making them intriguing candidates in brain or mental health biomarker discovery as well as intriguing candidates for therapeutic delivery systems. Early researchers believed EVs engaged only in removal of cellular wastes. However, recently researchers have begun unveiling their physiological and pathological roles. The therapeutic potential of EVs has been spotlighted due to their composition reflecting that of the parental cells.

In various embodiments, extracellular vesicles, including exosomes and exomeres, are harvested from normal or cancerous cell lines, stem cells, and cells produced in bioreactors, as well as mimetic nanovesicles as delivery agents. The extracellular vesicles can come from not just human but any mammalian, non-mammalian, bacterial, plant, fungal, yeast, algae, etc. sources. In various embodiments, drug-loaded EVs for cosmetic treatments, such as anti-aging therapies, may comprise EVs obtained from plant sources.

There are many ways to generate exosomes. One non-limiting method is to grow cells in culture, as the exosomes will be generated naturally and will be secreted into the media. The exosomes are harvested by collecting the media and then using the following extraction methods as outlined in the extraction protocols in reference.(⁶)

### Finding the Best Source of Parental Cells

Since exosomes are secreted by virtually all cells in all life forms, there is a wide selection of sources to work from. With respect to biomimicry and preserving what is already existing in nature, phytocannabinoids closely resemble endocannabinoids. Since endocannabinoids like anandamide (N-arachidonoylethanolamide, AEA) and 2-arachidonoylglycerol (2-AG); are formed by neurons and glial cells, it would make sense to find neurons that are easily cultured and have already been used as a base for extracellular vesicle harvesting. Microglial cells have been cultured in the past and have already been used to culture exosomes.^{(7,9)} The rationale for this approach is that exosomes generated from this cell line may mirror that of the cell, which would have proteins accessorized on the surface of the exosomes that would best target the cells that have the CB1 receptor on its surface. A method for culturing this particular cell line is described in the literature.⁽⁷⁾ A method for exosomal extraction using this cell line has also been described:⁽⁹⁾ Exosomes participate in signaling in the brain. Thus, it is rational to believe that endocannabinoids travel in extracellular vesicles naturally, as described in the literature:⁽¹⁴⁾

In view of the proposed mechanism of action of phytocannabinoids, it follows that the endocannabinoids (e.g., AEA, 2AG) would be travelling in an EV, (most likely in an exosome), in the natural pathogenesis of pain following an inflammatory disease.⁽¹⁾

Furthermore, with respect to phytocannabinoids that predominantly bind to the CB2 receptor such as CBD, and with respect to biomimicry, an immune cell can be used as an appropriate parental cell candidate for exosome harvesting, as most likely proteins and receptors accessorized on exosomes generated from this cell line would most likely target the CB2 receptors on immune cells (macrophages).

Mimetic nanovesicles: Although EVs have promising therapeutic potential, there are several challenges associated with using EVs before transition from the laboratory to clinical use. Some of these challenges include issues around low yield isolation and purification methods and poor engineering (loading) of EVs with therapeutic cargo. EVs architecture and cargo may need to be manipulated prior to clinical application, Some of these issues have been addressed by developing biomimetic EVs. EV mimetic-nanovesicles (M-NVs) are a type of artificial EVs which can be generated from all cell type with comparable characteristics as EVs for alternative therapeutic modality. There are a variety of these techniques usable to generate a natural phytocannabinoid mimetic based nanoparticles as well for the purpose of this disclosure.⁽⁸⁾

Other cell sources are useful for providing exosomes useful for bioactive loading in accordance with the present disclosure. These include, stem cells, cancer cell, myeloid cells, brain cells, bioreactors, and exosomes from plant, fungal or bacterial cells. Also, depending on the application, either for pain management, inflammation, neurodegenerative, autoimmune, cancer or recreational applications, the source of exosomes may vary as well as the desired cells to target will vary depending on the application and disease.

For example, in the case of inflammation, one might want to target a monocyte derived myeloid cell as they represent a large pool of scavenger and potential effecter cells during the inflammatory process, and uncontrolled activation of myeloid cells can lead to chronic inflammation. Myeloid cell mediated inflammation has been shown to play a major role in chronic illnesses. Furthermore, myeloid cells have a high capacity to take up vesicles like exosomes circulating in the peripheral blood. The same would be the case for pain management, and targeting the CB1/CB2 receptors for THC and CBD respectively.

For example, in the case of exosomal THC, one would want to target the CB1 receptors found in the brain. This might entail the choice to not only target brain cells, but to first attempt to harvest exosomes from cultured brain cells as well.

### Compositions, Dosage Forms and Methods of Administration

In addition, aside from using phytocannabinoids as the bioactive substance, therapeutic agents that are all nature-based, originating from plant, bacterial, fungal, yeast, mammalian, non-mammalian, algae, etc., i.e., any bioactive agent as defined above, can be used. In various embodiments herein, one can also use the methods herein in a precision based approach in the development of therapeutic formulations targeted not just for prevention and diagnostics, but as therapies for a host of diseases, such as, for example, cancer, mental illness, neurodegenerative diseases, autoimmune diseases, epilepsy, pain management, inflammation, etc. In other words, employing the methods described herein provide a universal diagnostic, prognostic, and therapeutic system to all living things in bringing the health of an organism to a homeostasis. With the advancement of bioinformatics applied to precision based medicines, the methodologies described herein are integrated to achieve an end product that is therapeutic, diagnostic, and prognostic for the organism being tested or treated.

In various compositions herein, EVs become the delivery vehicles for THC, CBD, other phytocannabinoids, endocannabinoids, terpenes, and any natural plant, fungal, bacterial, and mammalian compounds having anywhere from clinical therapeutic significance to homeopathic value to traditional beliefs. In various embodiments, EVs for the delivery of THC, CBD, other phytocannabinoids, etc., may comprise exomeres, such as those secreted by cancer cells, which are smaller (<50 nm in diameter) and functionally distinct from exosomes.

In various embodiments, exosomes are loaded with a desired bioactive substance, e.g., a therapeutic, medicinal, homeopathic agent, and the resulting loaded exosomes are targeted to the desired location in the subject by composition dosage form and administration route where the vesicles can release their contents to the target of interest. Packaging phytocannabinoids inside of exosomes or other EVs overcomes the problem of degradation of phytocannabinoids. Further, the phytocannabinoids are concentrated in their vesicle package and thus able to travel to desired cannabinoid receptors. Not wishing to be bound by any theory, delivery of cannabinoids via loaded EVs may mimic the way endocannabinoids travel within the body naturally.

Besides being encapsulated within natural vesicles, the bioactive substances of the present disclosure may also be admixed, encapsulated, conjugated or otherwise associated with other molecules, molecular structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The bioactive substances for loading into EVs encompass any pharmaceutically acceptable salts, esters, or salts of such esters, or any other compound which, upon administration to a human or non-human animal, is capable of providing (directly or indirectly) the bioactive metabolite or residue thereof. Accordingly, for example, the disclosure is also drawn to prodrugs and pharmaceutically acceptable salts of the bioactive substances of the disclosure, pharmaceutically acceptable salts of such prodrugs, and other bioequivalents. In various embodiments, a prodrug of a phytocannabinoid may comprise the acid form (i.e., prior to decarboxylation).

A prodrug is a bioactive substance that is prepared in an inactive form that is converted to an active form (e.g., a drug or supplement) within the body or cells thereof by the action of endogenous enzymes or other chemicals and/or conditions. In various embodiments, a prodrug is loaded into an exosome or other EV for formulation into a composition.

The vesicle-based compositions of the present disclosure may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Vesicle-based compositions for topical administration may include transdermal patches, ointments, lotions, creams, gels, drops, suppositories, sprays, liquids and powders. Conventional pharmaceutically acceptable carriers, aqueous, powder or oily bases, thickeners and the like, may be necessary or desirable.

In various embodiments, Exosomal- or exomer-based formulations of decarboxylated phytocannabinoids in an inhalable device formulation may be just as, or even more effective as, smoking, but without the negative side effects. Further, vesicle packaged phytocannabinoids in their decarboxylated and active forms eliminate variability/inconsistencies in the decarboxylation afforded by burning plant leaves, and the dosage/delay issues seen in edibles comprising inactive cannabinoid carboxylic acids.

In various embodiments, vesicle-based compositions of the present disclosure may be administered topically as transdermal crèmes, nasally or mucosally via a non-aerosol sprayer, or pulmonary via a nebulizing device such as a vape-pen or rescue inhaler.

The vesicle-based compositions of the present disclosure, which may conveniently be presented in unit dosage form, may be prepared according to conventional techniques well known in the pharmaceutical industry. Such techniques include the step of bringing into association the bioactive loaded vesicles with the pharmaceutically acceptable carrier(s) or excipient(s). In general, the vesicle-based compositions are prepared by uniformly and intimately bringing into association the bioactive loaded vesicles with liquid carriers or finely divided solid carriers or both, and then, if necessary, shaping the product by addition of various excipients.

The vesicle-based compositions of the present disclosure may be formulated into any of many possible dosage forms such as, but not limited to, tablets, capsules, gel capsules, liquids, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Vesicle-based compositions of the present disclosure include, but are not limited to, solutions, emulsions, and foams. The pharmaceutical compositions and formulations of the present disclosure may comprise one or more penetration enhancers, carriers, excipients or other active or inactive ingredients.

Emulsions are typically heterogeneous systems of one liquid dispersed homogenously in another in the form of droplets usually exceeding 0.1 μm in diameter. Emulsions may contain additional components in addition to the dispersed phases, and the bioactive loaded vesicles may be present in either the aqueous phase, oily phase, or on their own as a separate phase. Microemulsions are included as an embodiment of the present disclosure. Emulsions and their uses are well known in the art and are further described in U.S. Pat. No. 6,287,860, which is incorporated herein in its entirety.

Vesicle-based compositions of the present disclosure also include liposomal formulations. As used in the present disclosure, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells. In various embodiments, bioactive loaded vesicles may be part of a larger liposomal structure. In other embodiments, liposomes may be incorporated inside vesicles, along with one or more bioactive substances in the liposomes, in the vesicles but outside the liposomes, or both.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The vesicle-based compositions of the present disclosure may also include surfactants. The use of surfactants in drug products, formulations and in emulsions is well known in the art. Surfactants and their uses are further described in U.S. Pat. No. 6,287,860, which is incorporated herein in its entirety.

In various embodiments, the present disclosure employs various penetration enhancers to affect efficient delivery of bioactive loaded vesicles. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that compositions are routinely designed according to their intended use, i.e., route of administration.

In various embodiments, vesicle-based compositions for topical administration include those in which the bioactive loaded vesicles of the disclosure are in admixture with a topical delivery agent such as any combination of lipids, liposomes, fatty acids, fatty acid esters, steroids, chelating agents, and surfactants. Lipids and liposomes include neutral (e.g., dioleoyl phosphatidyl DOPE ethanolamine, dimyristoylphosphatidylcholine DMPC, distearoylphosphatidylcholine) negative (e.g., dimyristoyl phosphatidylglycerol DMPG) and cationic (e.g., dioleoyl tetramethylaminopropyl DOTAP and dioleoyl phosphatidyl ethanolamine DOTMA.

For topical or other administration, bioactive loaded vesicles of the disclosure may be further encapsulated within liposomes or may form complexes thereto, in particular to cationic liposomes. Alternatively, bioactive loaded vesicles may be complexed to lipids, in particular to cationic lipids. Fatty acids and esters, pharmaceutically acceptable salts thereof, and their uses are further described in U.S. Pat. No. 6,287,860, which is incorporated herein in its entirety.

Vesicle-based compositions for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Oral formulations are those in which bioactive loaded vesicles of the disclosure are administered in conjunction with one or more penetration enhancers, surfactants and chelators. Surfactants include fatty acids and/or esters or salts thereof, bile acids and/or salts thereof. Bile acids/salts and fatty acids and their uses are further described in U.S. Pat. No. 6,287,860, which is incorporated herein in its entirety. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Bioactive loaded vesicles of the disclosure may be delivered orally, in granular form including sprayed dried particles, or complexed to form micro or nanoparticles.

Vesicle-based compositions for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

In various embodiments, vesicle-based compositions in accordance with the present disclosure comprise one or more chemotherapeutic agents loaded into vesicles. Examples of such chemotherapeutic agents include, but are not limited to, cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxyco-formycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in vesicle-based compositions of the disclosure.

### In Various Embodiments, the Compositions and Methods Disclosed Herein Address the Following Technical Problems and/or Market Needs

A). Bioavailability

The problem: As mentioned, bioavailability of phytocannabinoids through traditional formulations is poor. The current methods to administer CBD/THC is through oral, mucosa, pulmonary (inhale), and topical/transdermal. The bioavailability of CBD through these methods are the following: sublingual (tinctures); 13-19%, oral (edibles 6-20%); and intranasal 34-46%.⁽²⁾

The advantages to EV compositions: Exosomes provide a delivery system that mimic nature. Although there is no way to improve the bioavailability of CBD/THC in its native form, e.g., from the Cannabis leaf, implementation of a vesical-based composition may enhance bioavailability of CBD/THC through a more effective administration route, such as the intranasal pathway. The reason being is that exosomes are produced by cells naturally, and is most likely in humans acting as the primary and natural delivery system of endocannabinoids (arachnoic acid and 2-AG) to its respective cannabinoid receptor (CB1 or CB2).

Formulation of a drug that would mimic nature in terms of its delivery system would be the most optimal in terms of bioavailability, as exosomes and or EVs are naturally produced and generating an exosomal formulation of CBD/THC would closely resemble that of nature compared with any other known formulations. This is a biomimetic innovation, therefore the implementation of phytocannabinoid formulation delivery system using exosomes would be expected to yield a final product that would have improved bioavailability and thus better efficacy, because it would closely resemble the process already naturally occurring in the endocannabinoid system. Also, due to the fact that the exosomes are exploited to carry 100% of their cargo as CBD/THC, the resulting loaded exosomes provide a delivery system that is more concentrated as compared to the current formulation which is a formulation that only carries a population of unprotected phytocannabinoids.

An analogy is to compare the phytocannabinoid to a passenger trying to cross an ocean (extracellular space) to get to a land destination (CB1 in neurons or CB2 in immune cells and macrophages) in order to do its job. If the phytocannabinoid were travelling on a cruise ship, or freight vessel, (exosome or other EV), protected by the elements, having all the amenities accessible to them, and leaving the responsibility of getting to its destination to the navigation system of the vessel it's travelling with, one can imagine the integrity of the phytocannabinoid would be much more conserved once it gets to its target destination as compared to the phytocannabinoid having to swim across the Atlantic ocean alone and unprotected. Taking it to a whole other level, imagine the cruise ship carrying over 5000 phytocannabinoids as passengers (in a vesicle). It is not difficult to imagine how effective the impact of the phytocannabinoids traveling in the ship collectively would be compared to thousands of individual phytocannabinoids that swam across the ocean unprotected (the current phytocannabinoid compositions herein). In the latter situation, only a very few would have been able to get to its destination, and out of those, very few would be able be intact, let alone functional. In this regard, it is not difficult to appreciate how leveraging the exosome or extracellular vesicle as a delivery system for phytocannabinoids would yield better formulations with regards to concentration, stability, drug target ability, efficacy, and bioavailability.

B). Degradation

The problem: CBD and THCs can degrade easily, decreasing the potency of the medicine. There are many factors that determining the shelf life of CBD and THC, such as the extraction methods, carrier oils used, etc. Also, how the product is stored also plays a significant role in storage and shelf life (i.e., storage at higher temperatures will cause shorter shelf life)³. In general, CBG, Δ⁹-THC, CBD and CBC phytocannabinoid subclasses are biosynthesized in Cannabis plants, while the remaining 6 subclasses (Δ⁸-THCA, CBTA, CBNA, CBNDA, CBEA, and CBLA)⁽⁴⁾ are probably the result of decomposition either in plant or due to poor storage conditions following harvest.

The advantages to EV compositions: Due to their stability, exosomes protect their cargo against degradation and denaturation in the extracellular environment. Therefore, encapsulation of THC/CBD phytocannabinoids with exosomes protects the precious cargo from degradation and denaturation from the outside elements, especially those present once administration into the body to shuttle the cargo to its target as safely as possible.⁽⁵⁾

C). Drug Reproducibility:

The problem: Because natural medicines are extremely difficult to reproduce due to the fact that the ultimate source is an organism, who's overall state can vary due to a myriad of factors such as environmental, chemical, genetic and a whole host of other variables, as well as batch to batch variability as compared to synthetic drug formulations, natural drugs are very difficult to reproduce. Therefore, and thus generally stable and consistent formulations are correspondingly difficult to produce. Furthermore, especially in the case of phytocannabinoids, the entourage effect is a difficult to determine, and it is known that there are many other phytocannabinoids that play a synergistic role in aiding certain other phytocannabinoids with a particular therapeutic effect. There currently are many labs in Israel and Canada performing research to identify the entourage effect, or moreover, to determine what might be the minimum number of components generated by the plant that are yielding the maximum therapeutic effect. Investigations underway to determine this composition may be tangentially translated and adapted into the formulations in accordance with the present disclosure. The problem with formulations that come from natural sources such as plants, is that formulations are hard to replicate, there does not yet exist proper standardization of analytical and quality control methods in the quantification and elucidation of the metabolic profile of a specific formulation, and so batch to batch variability is a real problem. Furthermore, the stability and shelf life of these phytocannabinoids is very unstable as they commonly degrade into an oxidized form of the original compound.⁽⁴⁾ Degradation is a common problem that reduces the integrity of the phytocannabinoid and quantification of it. In general, endocannabinoids and phytocannabinoids are not stable and can easily break down, thus resulting in a decrease in therapeutic potential.

The advantages to EV compositions: By employing a delivery system for these natural compounds through the use of exosomes, concentrations of therapeutic agent such as the phytocannabinoid can be measured and regulated through analytical equipment. Although the same testing is done with the current formulations, but due to the nature of implementing a delivery system where the concentrations can be measured on a per vesicle basis, some issues such as batch to batch variability can be overcome.

D). Adverse Side Effects with Current Formulations:

The problem: THC and side effects from smoking problem: How this may overcome that problem the route of administration of phytocannabinoids that are most effective is through inhalation (pulmonary) route. The main reason being is that this route does not go through first pass metabolism meaning it does not need to be digested and pass the liver prior to getting into the blood stream. By inhalation, the drug passes through the lungs and directly into the bloodstream and the effect is felt in seconds. Furthermore, the pulmonary method is the most effective. The problem is that in the case of THC and CBD, as with the other phytocannabinoids, they must first be decarboxylated in order to have a therapeutic effect. That entails smoking, which has signification adverse side effects, such as carcinogen exposure, difficulty breathing, chronic coughing, wheezing, and increased risk of lung cancer.

The advantages to EV compositions: By packaging a decarboxylated form of THC into an exosomal formulation in the design of an oralmucosal spray that can be ingested and directly enter the blood circulation system, bypassing the first pass metabolism path (digestion system, liver), one still benefits from the immediate therapeutic effect without having to inhale carcinogens from smoking Cannabis. Furthermore, the exosomal decarboxylated THC formulation would exist in particles no bigger than 150 nm, which can pass through the blood brain barrier and target the CB1 receptors located on the surface of the neurons in the brain.

E). The Opioid Crisis:

The problem: Because of the opioid addiction crisis, it has been determined that THC is a good candidate or alternative for pain management rather than opioid.

The advantages to EV compositions: Exosomal THC/CBD can be a better alternative for opioids for pain management. By creating an exosomal CBD/THC formulation which can be more stable and effective than the current native forms of THC/CBD, this may provide a better alternative to the current formulations, and as such, an even more powerful alternative to opioids for pain management. Thus, in various embodiments, THC and/or CBD-loaded EV compositions are used for pain management as an alternative to opioids.

### Examples

The examples delineated herein are not meant to be limiting. Additional experimentation is expected to optimize compositions for a particular bioactive substance.

Furthermore, depending on the therapeutic or homeopathic application of vesicle-based compositions, formulation methodologies may vary as well. All protocols mentioned below can be used in further developmental research to produce the best formulation for the specific bioactive and application.

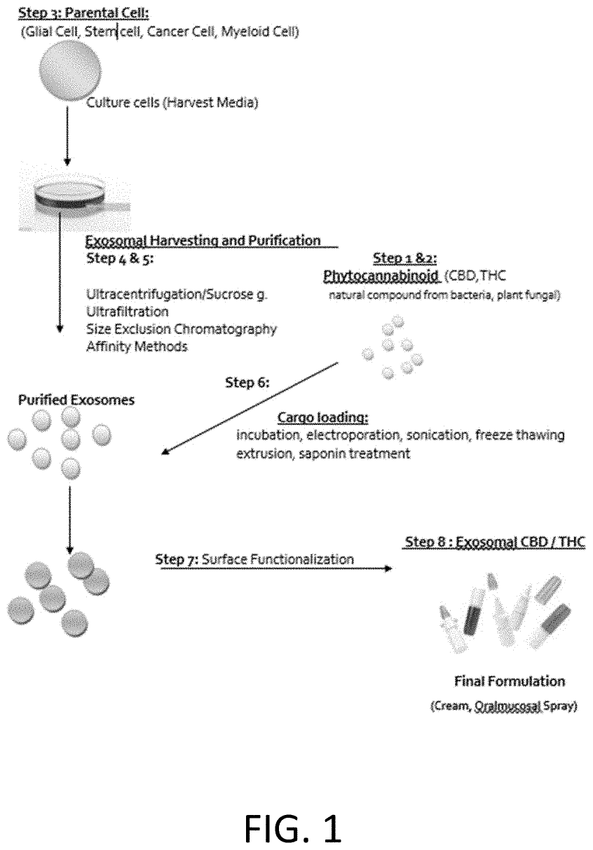

The general example delineated below is illustrated as a flowchart in FIG. 1. A variety of protocols and methodologies can be used to optimize the best final product, in terms of concentration, purity, stability, bioavailability and efficacy. Although specific examples may disclose cannabinoid loaded vesicle compositions, the present disclosure is not limited in the scope of the bioactive substance or substances loaded into vesicles, the type of vesicle, or the nature of the composition incorporating bioactive loaded vesicles.

### Step 1: Source and Preparation of Phytocannabinoid Bioactive Substance

In various embodiments, a crystalline form of THC/CBD is used for loading into the exosomes. The source of THC/CBD can be a reputable Cannabis manufacturer, having standardized analytical methods for culturing, extraction protocols, final formulation, characterization, detection, and contamination. Other sources of phytocannabinoid can be used as well, such as oils, tinctures, etc.

### Step 2: Decarboxylation

In order to activate the phytocannabinoid, the phytocannabinoid needs to be decarboxylated, which is achieved by heating the THC/CBD at specific temperatures. A decarboxylation protocol usable herein is disclosed in the literature.⁽¹¹⁾

### Step 3: Preparing the Exosomes

Various ways to generate exosomes are discussed above. For example, exosomes for use herein are released by autologous macrophages, such as RAW 264.7 macrophages in a conditioned media. In other examples, exosomes may be isolated from raw milk.

### Step 4: Isolation of Exosomes

The exosomes are extracted from the cell culture media using the methods described in the literature:⁽⁸⁾ Other methods can be employed, such as engineering the parental cells to modulate EVs as described in the literature:^{(8, 12)} In various embodiments, tiered centrifugations can be used. For example, centrifugation at 100,000×g for 60 minutes removes larger microvesicles whereas centrifugation of the resulting supernatant at 135,000×g for 90 minutes can be used to obtain a pellet of exosomes. Size based procedures are also used to isolate exosomes, wherein a filter having a pore size that enables isolation of the desired extracellular vesicle type (e.g., exosomes) is employed.

### Step 5: Purification of the Exosomes

The following purification methodologies can be used. These methodologies can be optimized as needed for a particular type of exosome.

Ultracentrifugation/Sucrose density gradient: The most standard methodology used for exosome purification is through ultracentrifugation followed by a sucrose density gradient application.

Size based applications: Although ultra-filtration through a 200 nm filter, such as to isolate exosomes as per above, may ensure better purity of the exosomes thus obtained, such a procedure has been known to be quite disruptive to the structural integrity of the exosomes. As an alternative, samples are passed through a size exclusion chromatography column (the qEV provided by iZON® Science Ltd.) in elevated heat (37° C.).

Affinity methods: Following the crude purification schemes as indicated above, and depending on the known properties of the desired exosomes, an affinity method can be applied. In various embodiments, a magnetic bead apparatus is employed comprising a canonical biomarker of exosomes such as CD9 (which seems to be exclusively accessorized on exosomes¹⁰ covalently attached to the magnetic bead), allowing to mix for some time, at some optimal temperature, and then a centrifugation step and a series of wash steps to remove the contaminants. Exosomes of interest are isolated following a step to detach the bond between the bead and CD9 ligand. Conversely, an affinity method is employed tailored to isolate the contaminants (lipoproteins, aggregates, etc.), in which the purified EVs would pass through the affinity column as the eluant or collected in the supernatant of the centrifugation step as in the case of an affinity method using magnetic bead, eliminating the step of having to dislodge the desired material from the affinity matrix.

### Step 6: Methods of Loading CBD into the Purified Exosomes

Incubation: In various embodiments, CBD loading is accomplished by incubation of the exosomes with CBD in its crystallized form in a buffer such as PBS as described in the literature.⁽⁶⁾ It is demonstrated in this study that exosomes form a complex with curcumin, which has similar characteristics as CBD/THC and hydrophobic and has poor solubility.

Other methods: Other more recent methods have been used to internally load cargo into exosomes and these additional methods find use herein. Some of these methods include electroporation, sonication, freeze thawing, extrusion and saponin treatment.⁽¹²⁾ Some other methods described in the literature can be employed.⁽⁸⁾

The amount of loading of bioactive substance into vesicles such as exosomes depends to a large extent on the nature of the bioactive substance, the vesicle-based composition and the administration route. A method of determining the amount of bioactive substance successfully loaded into vesicles is high performance liquid chromatography (HPLC). For some bioactive substances, the efficiency of loading into exosomes is ranked in the order: incubation<electroporation<<sonication, however this trend may be different depending on the nature of the bioactive substance, particularly its lipophilicity.

In various embodiments, bioactive loaded exosomes may be referred to as “ExoBioactive.” So, for example, THC loaded exosomes may be abbreviated as “ExoTHC.”

### Step 7: Surface Functionalization

In various embodiments, methods for accessorizing the surface of the exosomes boost targetability to get to the target cell/receptor, protein, etc. Some of these methods are highlighted in the literature references.^{(8, 12)} The phospholipid bilayer structure of the exosome enables direct incorporation of hydrophobic molecules to the EV surface, during the incubation phase of this method, providing target surface functionalization for phytocannabinoids.

Step 8: Formulating the Final Compositions

For the case of an exosomal phytocannabinoid product like exosomal CBD/THC as a therapeutic agent for inflammation, the final physical form for the vesical-based composition may be a topical/transdermal cream, or a composition for nasal/mucosal administration. The choice of a topical cream is ideal because clinical trials may be performed relatively easy since it is not as invasive an administration as injection or infusion. However, the best formulation may be an oral mucosal or nasal spray as this route of administration is through Mucosa Drugs. Cannabinoids, that are metabolized by liver and gut enzymes (first-pass hepatic metabolism), have specific pharmacokinetic requirements, demonstrate poor gastrointestinal permeability and cause irritation and therefore require alternatives to systemic oral delivery. Transdermal, nasal, inhaled-pulmonary and oral transmucosal delivery formulations enable drug uptake directly into the blood, thereby eliminating first-pass metabolism. The development of the transmucosal dosage form has provided a non-invasive method of administration that has proven itself to be significantly superior to oral dosage in the relief of pain (e.g., oral morphine vs. transmucosal fentanyl).⁽¹⁾

### II. Surface Modified Extracellular Vesicles

In various embodiments, surface modified EVs are disclosed. The EVs may be derived from mesenchymal stem cells (MSCs), which, when surface modified, find use in the treatment and prevention of various infections such as SARS-CoV-2/COV19 infections in the human and in non-human animal patients. In various embodiments, a surface modified EV comprises an EV with a biomolecule bonded thereon, the biomolecule comprising at least one of a polypeptide, protein or antibody recognizable by the ACE2 receptors in a patient. EVs from MSCs can also be used for therapies toward many diseases, such as of the intestine, kidney, lever, heart, and blood, and also for treatment of cancer and neurological diseases.

### Specificity for a Conserved Region

In various embodiments, it would make sense to target a more conserved region and mechanism, i.e. the spike protein on the surface of the viral particle as well as the mechanism of how the virus inserts its RNA into the host cell respectively. The main reason why the coronavirus (SARS-CoV-2) is so infectious is that the spike proteins located on the viruses' surface target the ACE2 receptor, which is abundantly located on the surface of lung epithelial cells (as well as kidney, intestine, heart, liver and neuronal epithelial cells). By targeting the specific binding site of the spike protein with the ACE2 receptor, with either an antibody or “plantibody” (or a peptide that binds to the specific region) that our body recognizes (at least with minimal side effect to host cells), we can block the virus from transferring its RNA into the host cell, thus blocking infection. In addition, to serving as the target entry port for the SARS-CoV/COV-19 viruses' RNA to get access to the host cell's photocopying machinery, the ACE2 receptor enzyme has a protective function for the lungs. Therefore, it is important to be mindful in targeting this enzyme such that the protective function is not lost as a consequence of inhibiting the virus's infection mechanism. There are other methodologies described below that one might employ by way of surface modification of the MSC-EV to target the virus/pathogen such that the pathogen is unable to bind to the host cell and inject its RNA for replication inside the host's cell.

In various embodiments, surface modified EVs provide a method of preventing the onset of various infections. For example, if the medicine is taken before infection, it could result in a protective/preventative effect as the surface engineered MSC-EV would bind to the surface receptor, inhibiting the virus from being able to get access to the site of infection. In addition, creating a formulation that entails extracellular vesicles (including exosomes and/or microvesicles) with a peptide that can specifically target the ACE2 receptor will not only inhibit the pathogenesis of the virus, but would yield a protective effect on the lungs due to the natural therapeutic effect of MSC-EVs.

In addition to targeting the ACE2 receptor, other targets could be used that would be engineered on the surface of the extracellular vesicles. For example, a surface peptide that would bind to the spike protein (as the spike protein is conserved amongst the generations of SARS-CoV), this may also block the ability of the virus from being able to bind to the host ACE2 receptor, as its spike protein would be blocked. Another methodology one could use is target TMPRSS2, which is the enzyme required to cleave the Spike protein on the virus surface, which enables the Spike protein to bind to the ACE2 receptor and insert its RNA. Possible mechanistic targets are found in the literature.⁽¹⁵⁾

### Time

In the current situation of rapid infection of the SARS-CoV-2/COV-19 (due of its mechanism of infection and because it targets something so abundant on the surface of the host organs), the development of vaccines is a time consuming process, allowing for a pandemic phenomenon which is the current situation (as mentioned above). Unfortunately, in a pandemic situation as we are currently facing, by the time a vaccine is created, the cost the damage the virus has created (physically, economically, socially, etc.) is simply too great.

As a solution to this problem, use of a medicine as described above overcomes the need to continuously generate antibodies because the surface modified EVs would target the very site that is required for SARS-CoV2 inject its RNA into the host cell to highjack the host cell's photocopying mechanisms. This treatment would be able to be administered immediately and would take effect quickly. In addition, there would be no need to generate a second generation type of formulation to recognize a mutated version of the previous virus due to the fact that the formulation described above is targeting a very conserved region on the coronavirus surface (S protein), as the “viral cloak” of coronaviruses do not appear to change.⁽¹⁵⁾

### Transmission Rate

The current average transmission rate (R0) is somewhere between 1.5-3.5, although it has been indicated these numbers are difficult to determine with much certainty.⁽¹⁶⁾ Most likely, the most important measure of the severity of the current SARS-CoV-2 pandemic is not only RO but the ratio of how many infected to how many die from this infection. Because of the current outbreak's high infection rate, and the number of patients that respond severely to the COV19 disease, having limited medical resources and health care to treat those patients who react severely to the infection, health care workers have to take on the daunting task of deciding who will live and who will die. It is for this reason that draconian social distancing rules and business shutdowns have been enforced in the hopes of preventing these situation.

Expecting the surface modified EVs to be successful in inhibiting the SARS-CoV-2 virus particle from binding to the ACE-2 receptor, the surface modified EVs would most likely reduce the RO or rate of transmission. There have been a number of studies that demonstrate both in vivo and in vitro that overexpression of ACE2 receptor was directly proportional to the severity of infection of SARS CoV-2.⁽¹⁶⁾

### Cytokine Storms and Ultimate Death

Unfortunately, because ACE2 receptors are abundant on the surface of epithelial cell which are on the surface of many host organs and thus is the route of how the coronavirus infects once it gets inside the host organism, the severity of infection is usually intense, the viruses' ability to copy itself and release the new copies through exocytosis of the host cell, the innate immune system gets turned on and can get hyperactive via generating a copious amount of cytokines alerting the immune system to go to war, long before our adaptive immune system (which has a better sense of “self” versus “non self”) kicks in, resulting in non-specific killing sprees of our own tissues, as in the cases of autoimmune diseases that could lead to irreparable damage and death.

By using the surface modified EVs above, one could prevent this (through the mechanisms described above) but in addition to that, Mesenchymal Stem cell Extracellular Vesicles (MSC-EVs) play an immunomodulatory role on lung epithelial cells by reducing the expression of inflammatory cytokines such as TNFα and IL-6. This medical invention could prevent not only infection from the virus, and thus could prevent a potential cytokine storm if taken post infection. Furthermore, it may also treat and reduce the cytokine storm already present in acute COV-19 patients already undergoing a cytokine storm condition, to reduce the expression of inflammatory cytokines, as well as save lives.

### Irreversible Damage Tissue Post Infection

Because of those patients that experience acute COV-19 infections leading to a cytokine storm like response where their immune system wreaks havoc its host, they undoubtedly may experience some irreversible damage due to the war and battlefield created by the hosts innate immune system.

Mesenchymal Stem cell Extracellular Vesicles (MSC-EVs) have therapeutic properties through the transfer of its mRNA and miRNA, proteins, (suspecting that mitochondria transfer is occurring perhaps via micro vesicle population to aid in oxygen transfer), as well as other components that repair damaged cells through cell growth, reduction of apoptosis (cell death), and a protective effect through the inhibition of ROS (reactive oxidative species), as well as providing oxygen to the tissues needing repair.

### Face Masks

Currently, the best way to reduce transmission (aside from social distancing) is to wash hands and wear face masks. Face masks are difficult to find (the best ones contain the

“N95” filter that can filter out virus particles) and there a shortage of masks. Furthermore, one needs to master the skill of handling and cleaning the masks, and without the proper skills, one may actually enhance the transmission of the virus if not used properly. Also, wearing masks is not very physically appealing. It is certain that individuals do not want to be wearing masks every time leaving the house for the next 2 years or until this pandemic gets under control.

By using an oral mucosal spray that coats the lining of mucus membranes and lung epithelial cells, if the virus can get that far, such as by addition of a viral deterrent for the mucous membranes, in view that there might be ACE2 receptors in the lining of the nose or mouth, it may not be so critical to use a mask, and would be more aesthetic to finally be free of masks.

### Storage, Shelf-Life and Preservatives

Vaccines are not very stable and as such need components like formaldehyde as preservatives. Cellular formulations need toxic formulations such as DMSO, which have been known to be toxic. Extracellular vesicles are very stable, can be frozen for 6 months without any preservatives their physical or functional integrity in still maintained. Thus, there is no need for toxic preservatives as in many other formulations as in the case for vaccines and cellular formulations.

### MSC-EVs Provide Better Formulation than MSCs

Despite the fact that MSCs used in clinical trials are efficacious, MSCs do possess the ability to promote growth of tumors and this property requires further research. MSCs have also been shown to become antigen presenting cells, expressing MHC II antigens, potentially causing immune responses in patients, which may exacerbate the condition.

MSC-EVs are non-replicating, reducing the risk of tumor formation, can be stored without DMSO at −80 C and remain biologically active. MSCs do not express MHC I or II antigens, are less susceptible to damage by hostile environments, at the site of injury, can get beyond the first capillary bed, whereas MSCs cannot.⁽²⁶⁾

In various embodiments, MSC-EVs have the following advantages over the “state of the art”: a robust, easy to use device to potentially prevent and treat SARS-CoV-2/COV19 patients, reducing transmission, preventing the likelihood in the event that there is an infection of an over reactive immune response or “cytokine storm” response phenomenon, that could potentially lead to death.

Because mesenchymal stem cell derived extracellular vesicles themselves can be therapeutic targets for a variety of diseases, the procedure of how one may employ creating a formulation that would target each type of disease could be universal, in terms of the research and development involved. Steps are provided below in the Examples for each disease where MSC-EVs can be used as a treatment and/or preventive.

### Examples

1. Culture MSCs

Choosing the proper mesenchymal cell line to harvest extracellular vesicles for optimal results: This will entail some research into which cell line to choose depending on the disease one would want to treat. For example, (ref 18), there are a variety of sources for MSC exosomes and their respective function in treating specific diseases, thus one could use the following cell lines for initial development, or as described in the literature.⁽¹⁷⁾

For asthma or diseases of the respiratory system (SARS-Co-2/COV-19): One would use a human bone marrow MSCs, or human umbilical cord derived MSCs, or adipose derived MSCs.

For diseases in the intestine (Inflammatory Bowel Disease): Bone marrow MSCs

For diseases of the liver: one would use MSCs from umbilical cord, HuES9.E1, adipose-derived MSCs.

For diseases of the heart: adipose—derived MSCs, human mesenchymal stem cell, bone marrow derived MSCs, HuES9.E1 MSCs, endometrium-derived MSCs.

For diseases of the kidney: human umbilical-cord derived MSCs, bone marrow MSCs, adipose tissue derived autologous MSCs.

For neurodegenerative diseases: human/mouse adipose—tissue derived MSCs, human mesenchymal stem cells, human/mouse bone marrow MSCs.

For Metabolic Diseases (Diabetes): human multipotent stromal cells.

### Methods to Grow MSCs from Culture

Previously described methods may be used.⁽²⁷⁾

In various embodiments, parental mesenchymal stem cells may be stimulated with proteins or genetic material by adding to the media during culturing of the cells to generate a desired exosomal/EV formulation (i.e. cargo composition and surface expression that would serve a therapeutic/anti-inflammatory properties), as an alternative to loading the exosome cargo/surface modification post EV harvesting/purification. A brief description of this methodology can be found in the literature.⁽²³⁾

### Large-Scale Production

It has been mentioned there is still a requirement for optimization of methodologies for harvesting; however, a possible method for harvesting Mesenchymal Stem

Cells is outlined in: “Largescale EV production employs the use of large or multi-layer culture flasks, fixed-bed bioreactors, in-stirred tank bioreactors, or continuous production in perfusion reactors,” (Colao et al., 2018).⁽¹⁹⁾ It is known that there will be hurdles in the step of mass production of MSC derived EVs as the efficacy of the therapeutic effect of extracellular vesicles from MSCs might be lost due to scale up procedures. One may be able to overcome this hurdle through experimentation targeted at better determining the composition/cargo components of the exosomes/EVs that are causing the therapeutic effect, and then mass producing another EV or extracellular vesicle that can be mass produced, and packaging EVs from that origin with the components that have the medicinal properties.

2. Extraction of EVs from MSCs:

The EVs could be extracted from the cell culture media using literature methods.⁽²⁰⁾ Other methods could be employed such as engineering the parental cells to modulate EVs.^{(20,23)} Independent of the disease one would want to employ to treat, the extraction methodologies would be relatively similar. Large scale methods would also be implemented once the optimal source has been determined. There are many steps one might take in harvesting EVs from MSCs.

3. Purification of Exosomes:

The purification methodologies would need to be explored and optimized depending on the population of EVs one would want to target. please find below a list of possible extractions and purification methods:

3a) Ultracentrifugation/Sucrose Density Gradient:

The most standard methodology used for exosome purification is through ultracentrifugation followed by a sucrose density gradient application.

3b) Size Based Applications: Although one may also use ultra-filtration method through a 200 nm filter to ensure better purity, this has been known to be quite disruptive to the structural integrity of the exosomes. It would be advantageous to pass the samples through a size exclusion chromatography column (the qEV provided by iZON Science Ltd.) in elevated heat (37 C) might yield optimal results.

3c) Affinity methods: Following the crude purification schemes as indicated above, and depending on the known properties of the desired exosomes, an affinity method could be explored. One could apply a method that would employ a magnetic bead contraption that would have some kind of canonical biomarker of exosomes like CD9 (which seems to be exclusively accessorized on exosomes⁽²²⁾ covalently attached to the magnetic bead, allowing to mix for some time, at some optimal temperature, and then a centrifugation step and a series of wash steps to remove the contaminants. One would need to isolate the exosomes of interest following a step to detach the bond between the bead and CD9 ligand.

4. Packaging the MSC-EVs and Determining The Optimal Cargo Composition:

4a) Determining the Optimal Cargo Composition:

Because it has been highlighted in numerous papers that there is growing evidence that Mesenchymal Stem Cell (MSC) based therapies are mainly attributed to the effects of MSC-derived extracellular vesicles,⁽²¹⁾ these extracellular vesicles appear to naturally have a therapeutic effect without changing or having to package the exosomes/EVs for use as delivery agents only. Therefore, more research and development will be required to elucidate precisely the EV composition that is providing the therapeutic effect for a particular given disease, prior to embarking on the EV packaging step. Most likely, in the case of exosomes, it is a mRNA or miRNA, (that reduce inflammation and cell apoptosis), or a combination thereof that is providing the therapeutic effect.

4b) Possible Clues/Directions for EV Packaging:

For example, it has been determined that specific MSC EVs target specific pathways for a specific disease⁽¹⁸⁾ as illustrated in the drawings. This can provide an idea of what pathways are targeted, and therefore the possible agents that one might choose to package MSC-EVs. Some other ways in determining the composition of MSC-EVs can be through the use of technologies and methodologies in the literature.⁽²⁸⁾

4c) Disease, Targets, and Pathways—Possible Packaging Components:

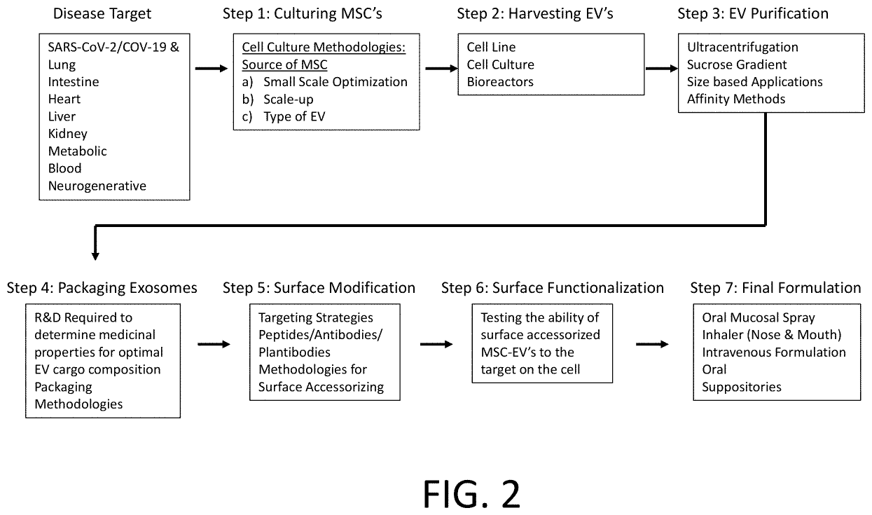

FIG. 2 sets forth a flowchart of steps to obtain a final formulation comprising drug-loaded EVs beginning with a “disease target.” In various embodiments, the disease target is a SARS-CoV-2 infection in a human individual, in which case the drug active packaged into the exosomes (Step 4) may comprise the REGN-COV2 antibody cocktail, or remdesivir or another antiviral active agent. As illustrated in FIG. 2, the disease target may comprise both a disease (like a cancer, or an infection) and also a target organ or physiological system. Once the disease state is identified, Steps 1, 2 and 3 comprise obtaining EVs that are appropriate for the actives to be used for the disease target. It should be noted here that “disease” is used broadly in the context of FIG. 2, and includes even aches and pains and cosmetic issues. As shown, Steps 1 and 2 include various methods of obtaining EVs, and Step 3 sets forth some nonlimiting options for purifying the EVs of choice. Step 4 comprises the loading of drug actives into the EVs. Steps 5 and 6, comprising methods for modifying and functionalizing the surfaces of the EVs are optional. Step 7 sets forth the various finished drug delivery forms that may be utilized once the drug-loaded and optionally surface decorated EVs are obtained.

### III. Novel Methods for the Identification and Isolation of EV Subsets

In various embodiments of the present disclosure, novel and inventive methods are described for the identification and isolation of EV subsets. In various embodiments, such methodologies comprise identifying and isolating a population of EVs from natural sources that are shown to possess a specific therapeutic effect for a specific targeted ailment or disease. Having identified and isolated specific EV subsets in this way will lead to a) improved natural therapies; b) an improvement in understanding the “entourage effect” of medicinal plants; c) an advancement of AI and computational methods using “omic” databases.

The basis of this methodology to identify and isolate EV from natural sources could lead to the identification of a complex of biomolecules capable of eliciting an “entourage effect,” and efficacy for a specific disease. Once biomolecules are identified, loading these biomolecules (e.g., through electroporation or simple incubation) into natural

EVs would lead to great therapies. Further, there are technologies that highjack EV genesis in the parental cells so that the parental cells will secrete EVs that contain whatever cargo desired.

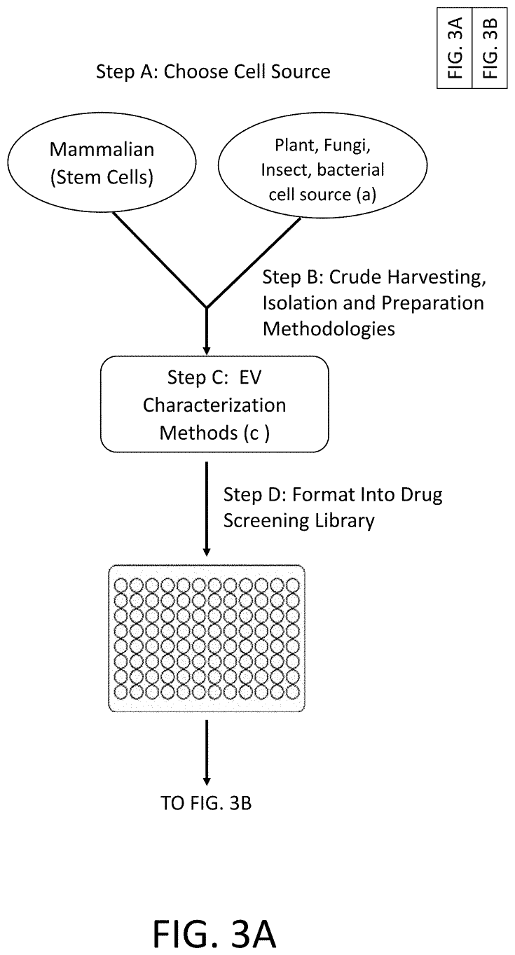

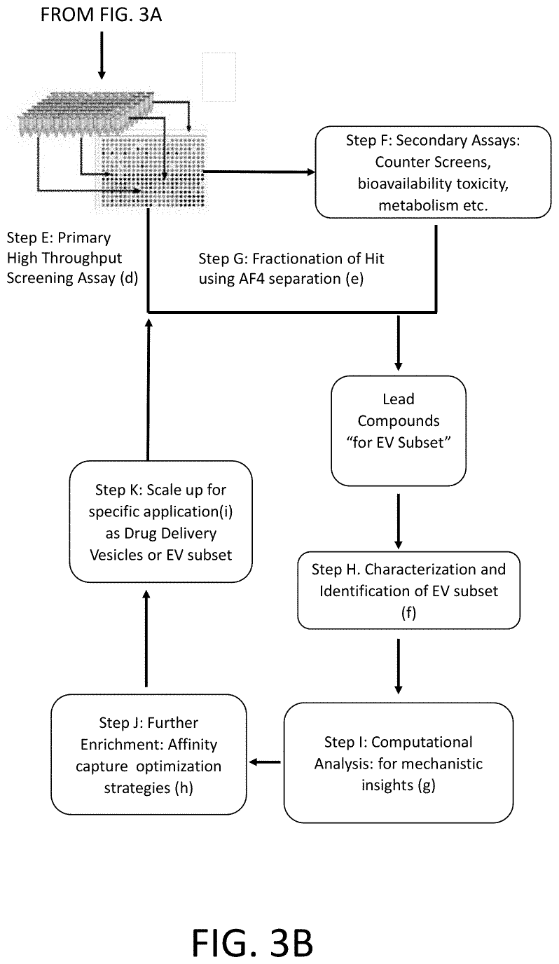

FIGS. 3A and 3B set forth a flowchart of steps used to identify and isolate an EV subset comprising naturally-sourced EVs. The steps comprise a broad road map on how to isolate and identify an EV subset for a particular desired outcome. In various embodiments, the method of FIGS. 3A and 3B comprise the steps of identifying EV subsets from natural or pristine sources, rather than bioengineered drug carriers (e.g., as outlined in the process of FIG. 1). The outlined approach will successfully isolate EV subsets from biological fluids.

With reference to FIGS. 3A and 3B, the schematic for isolating EV subsets from pristine/natural sources begins with Step A, choosing the cell source, e.g., based on evidence (like scientific evidence) that supports a particular cell source for a particular disease and/or output. As shown, cell sources may be either mammalian or plant, fungi, insect, bacterial, and the like. What is meant by “for a particular disease and/or output” is that the pathogenesis of the disease involves EVs, and therefore it is those EVs that are preferred for use in forming drug-loaded and optionally surface modified EVs for compositions to treat that particular disease. In some cases, the output is not a disease, but rather a condition, or ailment, such as pain.

In a specific example, a study has shown that extracellular vesicles are involved in the pathogenesis of rheumatoid arthritis and osteoarthritis.⁽³⁹⁾ Given this scientific evidence of the involvement of EVs in the pathogenesis of rheumatoid arthritis and osteoarthritis, the first step comprises choosing the cell source that produces these EVs in developing drug-loaded and optionally surface functionalized EVs for treatment of rheumatoid arthritis and osteoarthritis. The study suggests that EVs from IL-1β-stimulated fibroblast-like synoviocytes have been shown to induce osteoarthritic changes in chondrocytes. Therefore, it is these synoviocytes, cells that line the inner surface of joints and tendon sheaths, that are the chosen cells for the EVs. These EVs, once isolated, characterized, fractionated and identified, may be loaded with a drug active shown effective against rheumatoid arthritis and osteoarthritis, such as for example, sarilumab, a human monoclonal antibody that binds to the interleukin 6 (IL-6R) receptor. In this way, a pharmaceutical composition is created comprising sarilumab-loaded vesicles, wherein the vesicles are the same human extracellular vesicles that participate in the pathogenesis of rheumatoid arthritis and osteoarthritis. Such compositions may then be effective even if administered to the patient in need thereof by routes other than the usual injection administration for sarilumab (e.g., Kevzara® from Regeneron and Sanofi).

The next step in the process is Step B, the crude harvesting, isolation and preparation methodologies. Methodologies for isolation and preparation from pristine plant sources may involve, for example, grinding, differential centrifugation and sucrose gradient techniques.⁽³⁰⁾ A general protocol for culturing, harvesting and isolating EVs from mammalian cells and other sources have been described.⁽³³⁾

The next step in the process is Step C, EV characterization methods. Notably, plant and mammalian sources share mutual common techniques for characterization of cell-derived EVs. Characterization methods include, but are not limited to, atomic force microscopy (AFM), dynamic light scattering, flow cytometry, immunoblotting, immune-sorbent analysis, transmission electron microscopy (TEM), nanoparticle tracking analysis (NTA), scanning electron microscopy (SEM) or tunable resistive pulse-sensing. In various embodiments, characterization comprises immunoblotting of specific proteins known to be present on the surface of the EV of interest. The next steps are to format into a drug screening library (Step D) and to perform primary high throughput assays (Step E). A general methodology on how to design a high throughput screening assay is described in the literature, for both biochemical and cell-based screening assays.^{(35,36)} For example, if one were to screen for an EV subset originating from mesenchymal stem cell or other source as a drug candidate to treat SARS-CoV-2 infection, one could use a drug screening model system as outlined in the literature.⁽³⁷⁾

With continued reference to FIGS. 3A and 3B, Step F in the methodology is to perform secondary assays, such as counter screens, bioavailability, toxicity, metabolism, and other assays. Step G in the methodology is to fractionate, such as by utilizing an “asymmetric flow field-flow fractionation (AF4) instrument, configured for nanoparticle separations. Fractionation of plant library sources using AF4 technology is described in the literature.⁽³¹⁾ One may employ hydrophobic interaction chromatography as well.⁽³³⁾ One would employ this technique to optimize the fraction that contains activity, so as to identify the most concentrated population of desired EV subset for the given application based on the screening assay.

Step H in the flowchart is the characterization and identification of the EV subset. Various methodologies that can be applied to these techniques include:⁽³⁰⁾

1. Biochemical Characterization: Immunoblotting for specific proteins;

2. ELISA, SDS PAGE, Liquid Chromatography, microfluidic electrophoresis analyzer, high throughput small RNA sequencing, EV Flow cytometry, and GC/Mass spec: for deep analysis of EV subset components on cargo;

3. Bicinchoninic acid (BCA), fluorometric, SDS PAGE analysis: Protein content;

4. Raman spectroscopy: chemical structure and biomolecules;

5. Microarray analysis (EXOCHIP), digital droplet PCR. Next generation sequencing techniques: for RNA content of EV subsets; and

6. Sulfo-phospho-vanillin assay, total reflection Fourier transform infrared spectroscopy. Fluorescent dyes: quantification and lipidomic analysis.

Morphological characterization methods include:

1. TEM (Transmission Electron Microscopy): Size and Morphology, ultrastructure analysis;

2. NTA (Nanoparticle Tracking Analysis) Nanoparticle Concentration;

3. AFM (Atomic Force Microscopy): Structural and size related understanding of subset;

4. DLS (Dynamic Light Scattering): measures precis size, and evaluates size distribution; and

5. EV Identification: Lipids, Proteins, RNA, Metabolites.

With continued reference to FIGS. 3A and 3B, Step H in the methodology comprises computational analysis, such as for mechanistic insights. Computational Analysis^{(32,38)} in the form of Artificial Intelligence (AI), and a subset thereof termed Machine Learning (ML) are useful in a variety of applications for extracellular vesicles from biological fluids:

Diagnostics: The discovery of novel disease biomarkers for diagnostics (ex. Liquid biopsy), and subsequently predict the onset of a particular disease;

Prognostics, for prediction of the best therapy for a particular disease for a specific patient, and the ability to monitor the effectiveness of treatment in real time, through EV analysis;

Theragnostic: EV Computational Analysis could be applied in multitude of ways, one of which would be as an aid to elucidate cellular mechanisms and disease pathways for a specific EV subset. This would aid in the characterization of identification of therapeutic EV subsets;

Language interpretation (Enigma Machine): By using a combinatorial approach with AI and ML algorithms, EV Databases (e.g., EVmiRNA, EVpedia, EV-Track, ExoCarta, ExoRBase, Exosome Gene Ontology Annotation Initiative, Plasma Proteome Database, Urinary Exosome Protein Databases, Vesiclepedia), Microfluidics, EV Isolation techniques, and various “omics” (e.g., genomics, transcriptomics, metabolomics, glycomics, and lipidomics), EV identification/characterization methods one could distinguish the heterogenicity of EV subsets through defining a specific output. This could be the building block towards deciphering signal to noise, and thus a Rosetta stone to unlock this Universal life language that is coded within EVs. Efforts in this area will undoubtedly prove invaluable to better the health for not only humans, but all life forms. One such application would be the basis of designing a device that could analyze a patients EV profile through their biological fluids, and using computational analysis to match with the best EV subset (from any organism EV subset library) that would predict the likeliest outcome supporting homeostasis in the patient;

Affinity Assays: Once a specific EV subset is identified and characterized, one could develop affinity assays using antibodies or peptides that specifically bind to a surface protein/receptor/ligand. This, along with other previous isolation methodologies like Size exclusion Chromatography (SEC), centrifugation, filtration, to be optimized for purity and yield; and

Scale Up: Scale up methodologies are outlined, for example, in the literature.⁽³⁵⁾

Plants comprise a natural source for EVs.⁽²⁹⁾ To maximize the chances of finding an extracellular vesicle subset for a particular therapeutic agent, especially in the context of microbial, antifungal, and antiviral agents, the optimal source one would want to use would be located in the apoplast of plant leaves, where exosomes are deployed from plant cell as defense mechanism to stop pathogen invaders. In addition, one would expect that this intercellular communication mechanism between pathogen and host plant through the release of exosomes from both sources into the apoplast (battlefield), would be constantly evolving and upgrading their defenses to produce bioactive molecules through DNA, RNA, proteins, lipids, metabolites, and glycols in ways that they are naturally evolving to create optimized therapeutic molecules pathogen defense and repair of host cells. One could envision exosomes (or other EVs) extracted from the apoplast as nature's medicinal factory making machines. Hence, it would make sense that performing extractions in this area, along with the phloem of plants (as a secondary site), would be very fruitful in terms of the discovery of EV subsets of clinical relevance.

Diseases of the lung/Respiratory System Acute Respiratory Syndrome (ARDS) or viral infection and pneumonia via SARS-CoV-2 and the pathways that MSC-EVs trigger: Immunomodulatory effects: Suppression/Regulation of cytokines TGFβ, 1, IL-10. Inflammation via MiR-181c, TLR4.

Diseases of the Gut/Inflammatory Bowel Disease: miR-21, PTEN, miR181c, TL4.

Diseases of the kidney: VEGF, TNFα, IL-6, IL-18, and IL-1β, mRNAs, IGF-1R, Bcl-x1, Bcl-2, BIRC8, Casp1, Casp8, and LTA, lipocalin.

Diseases of the liver: TGF β1, /Smad2, Cyclin D1, Bcl-x1, STAT3, TNFα, INF-γ, IL-6, IL-18, and IL1β.

Diseases of the heart and blood: Wnt/β-Catenin, miR-21, PTEN, P13K, Akt, miR22, Mecp2, miR-125b, miR-21-5p, P13K, AMPK/mTOR, Akt/mTOR, miR-17-92.

Diseases of the neurodegenerative order: neprilysin, miR17-92, miR-133b, PTEN, rapamycin/glycogen synthase 3β signaling pathway, β amyloid.⁽17)

Disease of the metabolism (Diabetes): Th1, Th17.

5. Surface Modification of MSC-EVs:

5a) Surface Modification Depends on Disease to Treat/Prevent:

The surface modification of EVs would depend on the disease and hence the cell one would want to target. SARS-CoV-2: For surface Modification of SARS-CoV-2/COV-19 diseases: One could accessorize the surface of the EV via a number of ways. Here are a few (please see diagram 2 that illustrates possible targets):

Proposition 1: Target Spike Protein Binding Region on the ACE2 Receptor Protein:

(a) To generate the ACE-2 target: First, one could generate a peptide that recognizes the binding region on the ACE 2 receptor: “Wan et al. reported that residue 394 (glutamine) in the SARS-CoV-2 receptor binding domain (RBD), corresponding to residue 479 in SARS-COV, can be recognized by the critical lysine 31 on the human ACE2 receptor.⁽¹⁵⁾ A peptide generated to this region would be used.

(b) In addition, there are a number of ACE2 receptor antibodies that are commercially available (albeit it is indicated “for research use only”, there still needs to be work performed in finding an antibody that would be safe to use on humans. This will most likely be generated very quickly. One could generate an antibody that recognizes the binding region on the Spike protein or ACE2 receptor that the Spike protein binds to.

(c) “Plantibodies”: One way to generate an ACE-2 receptor antibody is through plants for the region described above. There are many advantages to using plants instead of mice and other animals. These antibodies are termed “plantibodies”, these can be made from transgenic tobacco plants, or another plant source.

Proposition 2: Targeting the Spike Protein (“Mop”)

In addition to generating peptides/antibodies/plantibodies that target the ACE 2 receptor, one could also generate a peptide/antibody/plantibody that targets the Spike protein. This could then act as an EV “mop” phenomenon whereby the accessorized EV could bind to the virus, making it incapable of binding to the ACE2 receptor and thus resulting in the virus to be unable to insert its RNA for replication.

Proposition 3: Generate an exosomal/EV population that would be able to specifically attack the virus, once the EV/exosomal composition, surface modification, and phenotype are determined.

Proposition 4: Other mechanisms may be employed using novel inhibitors of Severe Acute Respiratory Syndrome Coronavirus Entry that act by Three Distinct Mechanisms.⁽²⁵⁾

Other Diseases: In the case for other diseases, one could accessorize the MSC-EVs to target the following cells (of course, one would need to perform research in deciding what area to target such that one would know the proper peptide/protein/antibody to target.⁽²¹⁾

Diseases of the lung (In addition to SARS-CoV-2/COV-19: alveolar macrophages, lung DCs, neutrophils, monocytes.

Diseases of the kidney: Renal tubular cells, PTECS.

Neurodegenerative Diseases: Microglia, hippocampal neurons, CD+4T cells, neurons, macrophages.

Diseases of the heart: cardiomyocytes

### 5b) Accessorizing the Surface of the EV with the Peptide/ACE2 Receptor of Choice

There are many methodologies for accessorizing the surface of EVs: There are methods for accessorizing the surface of the exosomes to boost targetability to get to the target cell/receptor, protein etc. Some of these methods are highlighted in the literature references.^{(20,23)} Fortunately, it may be quite easy to target surface functionalization for because the phospholipid bilayer structure of the exosome enables direct incorporation of hydrophobic molecules to the EV surface, during the incubation phase of this method.

Other Methodologies: Other more recent methods have been used to internally load cargo into exosomes and could be also explored. Some of these methods may include electroporation, sonication, freeze thawing, extrusion and saponin treatment.⁽²³⁾ Some other methods described in the literature could be employed.⁽²⁰⁾

### 6. Surface Functionalization

There are methods for accessorizing the surface of the exosomes to boost targetability to get to the target cell/receptor, protein etc. Some of these methods are highlighted in the above references.^{(20,23)} Fortunately, it may be quite easy to target surface functionalization because the phospholipid bilayer structure of the exosome enables direct incorporation of hydrophobic molecules to the EV surface, during the incubation phase of this method.

### 7. Final Formulations

The final formulation would be determined based on the disease and most effective route of administration based on the cells that one would want to target. There undoubtedly would need to provide more research into the route of administration and thus formulation based on previous research as to what may work based on the drug/target/pharmacokinetics etc.

### In the Case for a Respiratory Disease such as the Current Pandemic of SARSCoV-2/COV-19:

One might use an oral mucosal spray or inhaler that would cover the nose and the mouth. This may entail generating an inhaler type device with a spacer. A detailed description of such devices is described in the literature.⁽²⁴⁾

For diseases of the intestines, blood, liver, kidney, metabolism; heart:

An intravenous device might be the most advantageous in this scenario. Other formulations may include an oral pill, or suppositories. For neurodegenerative diseases, it might be advantageous to use an oral mucosal spray/inhaler, as a primary choice to deliver MSC-EVs, or orally.

Other aspects of the formulation that would need to be explored:

In order to minimize the spread of entry of a virus such as SARSCoV-2/COV-19, if it is determined that entry of the virus is possible by the eyes or ears (orifices of the body), then a slightly different formula would need to be designed for eye drops and/or ear drops to minimize the ability of the virus to spread throughout the host organism. For example, a person with viral particles present on their hand may provide an entry point for the virus throughout their body simply by touching their eye or ear.

### Additional Considerations

In various embodiments, a composition comprises extracellular vesicles, the extracellular vesicles comprising at least one bioactive substance loaded therein.

In various embodiments, the bioactive substance comprises at least one of a phytocannabinoid or endocannabinoid. In certain embodiments, the bioactive substance comprises at least one of THC or CBD.

In various embodiments, the extracellular vesicles comprise exosomes.

In various embodiments, the composition is in the form of a liquid, oil, cream, or powder.

In various embodiments, a method of treating a disease in a human or non-human animal comprises administering to the human or non-human animal in need thereof a therapeutically effective amount of a composition comprising extracellular vesicles loaded with at least one bioactive substance.

In various embodiments, the disease comprises cancer, mental illness, a neurodegenerative disease, an autoimmune disease, or epilepsy.

In various embodiments, the bioactive substance comprises at least one of a phytocannabinoid or endocannabinoid.

In various embodiments, the bioactive substance comprises at least one of THC or CBD.

In various embodiments, the extracellular vesicles comprise exosomes.

In various embodiments, a method of managing pain in a human or non-human animal comprises administering to the human or non-human animal in need thereof a therapeutically effective amount of a composition comprising extracellular vesicles loaded with at least one bioactive substance.

In various embodiments, the bioactive substance comprises at least one of a phytocannabinoid or endocannabinoid.

In various embodiments, the bioactive substance comprises at least one of THC or CBD.

In various embodiments, the extracellular vesicles comprise exosomes.

In various embodiments, a method of treating inflammation in a human or non-human animal comprises administering to the human or non-human animal in need thereof a therapeutically effective amount of a composition comprising extracellular vesicles loaded with at least one bioactive substance.

In various embodiments, the bioactive substance comprises at least one of a phytocannabinoid and endocannabinoid.

In various embodiments, the bioactive substance comprises at least one of THC or CBD.

In various embodiments, the extracellular vesicles comprise exosomes.

In various embodiments, a method of promoting general health, or for reducing or delaying onset or recurrence of a disease, illness or condition, in a human or non-human animal comprises administering to the human or non-human animal in need thereof a prophylactically effective amount of a composition comprising extracellular vesicles loaded with at least one bioactive substance.

In various embodiments, the bioactive substance comprises at least one of a phytocannabinoid and endocannabinoid.

In various embodiments, the bioactive substance comprises at least one of THC or CBD.

In various embodiments, a method of manufacturing phytocannabinoid or endocannabinoid loaded exosomes comprises culturing parental cells capable of naturally producing exosomes; isolating the exosomes; purifying the exosomes; and incubating the purified exosomes in a buffer comprising the phytocannabinoid or endocannabinoid for a time sufficient to load the exosomes ex vivo with the phytocannabinoid or endocannabinoid.

In various embodiments, the phytocannabinoid comprises at least one of THC or CBD.

In various embodiments, a composition comprises extracellular vesicles, the extracellular vesicles comprising at least one biomolecule bound thereon.

In various embodiments, the at least one biomolecule is selected from the group consisting of nucleotides, nucleosides, oligonucleotides, DNA, RNA, hybridization probes, amino acids, polypeptides, proteins and fragments thereof, and antibodies and fragments thereof

In various embodiments, the at least one biomolecule targets an ACE2 receptor.

In various embodiments, the at least one biomolecule is a plantibody.

In various embodiments, a surface modified extracellular vesicle is used in the treatment or prevention of a SARS-CoV-2/COV-19 infection, the surface modified extracellular vesicle comprising mesenchymal stem cell derived extracellular vesicles further comprising a biomolecule attached thereon capable of binding to an ACE2 receptor.

In various embodiments, the biomolecule comprises an ACE2 receptor antibody or a polypeptide capable of hybridizing to a portion of an ACE2 receptor.

Bioactive-loaded extracellular vesicles, surface modified extracellular vesicles, and methods thereof, are generally provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that

A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for a composition or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

### Literature References

1. Cannabinoid Delivery Systems for Pain and Inflammation Treatment Natascia Bruni, Franco Dosio, Molecules, 25 Sep. 2018
2. https://www.cbdoil.org/cbd-bioavailability/3.
3. https://www.marijuanabreak.com/cbd-oil-shelf-life
4. “A new ESI-LC/MS approach for comprehensive metabolic profiling of phytocannabinoids in Cannabis,” Paula Berman 24 Sep. 2018.
5. http://www.weizmann.ac.il/Biomolecular_Sciences/Regev/research-activities/malaria-derived-exosomes
6. “A Novel Nanoparticle Drug Delivery System: The Anti-inflammatory Activity of Curcumin is Enhanced when encapsulated in Exosomes”, Dongmei Sun, Huang-Ge Zhang, www.moleculartherapy.org, September 2010.
7. https://www.ncbi.nlm.nih.gov/pmc/articles/PMC5669279/ Culturing Microglial Cells
8. “Methods for loading therapeutics into extracellular vesicles and generating extracellular vesicles mimetic-nanovesicles.” Andrew Hill, 2 Jan. 2020
9. https://pdfs semanticscholar.org/6c92/ec8d4be34be9582c3eb6049d998db2a0f350.pdf Extraction of microglial cells
10. “Physical and Molecular Landscapes of Mouse Glioma Extracellular Vesicles define Heterogeneity,” Cell Reports, Jun. 25, 2019
11. https://www.ncbi.nlm.nih.gov/pmc/articles/PMC5549281/pdf/can.2016.0020.pdf decarboxylation
12. “Engineered extracellular vesicles and their mimetics for clinical translation, Methods,” Hyejin Kim, 1 Aug. 2019.
13. https://www.grandviewresearch.com/press-release/global-legal-marijuana-market
14. “The emerging role of exosomes in mental disorders, Translational psychiatry, Saumeah Saeedi, September 2019. https://www.nature.com/articles/s41398-019-0459-9.
15. Angiotensin-converting enzyme 2 (ACE2) as a SARS-CoV-s receptor: molecular mechanisms and potential therapeutic target. Haibo Zhang, Intensive Care Med (2020).
16. https://labblog.uofmhealth.org/rounds/how-scientists-quantify-intensity-of-an-outbreak-like-covid-19
17. “Mesenchymal stem cell derived extracellular vesicles: novel frontiers in regenerative medicine”.
18. “Exosomes from mesenchymal stem/stromal cells: A new paradigm” Kan Yin 2019
19. “Mesenchymal Stem Cell-Derived Extracellular Vesicles: Challenges in Clinical Applications”, Austin Gowen March 2020
20. “Methods for loading therapeutics into extracellular vesicles and generating extracellular vesicles mimetic-nanovesicles. Andrew Hill, 2 Jan. 2020”.
21. “Mesenchymal Stem Cell derived Exosomes and Other Extracellular Vesicles as New remedies in the Therapy of inflammatory diseases” Carl Harrell, Cells 2019.
22. “Physical and Molecular Landscapes of Mouse Glioma Extracellular Vesicles define Heterogeneity, Cell Reports Jun. 25 2019.
23. Engineered extracellular vesicles and their mimetics for clinical translation, Methods Hyejin Kim, 1 Aug. 2019.
24: https://www.aarc.org/wp-content/uploads/2015/04/aerosol_guide_rt.pdf
25. Novel Inhibitors of Severe Acute Respiratory Syndrome Coronavirus Entry That Act by Three Distinct Mechanisms, Adeyemi O. Adedeji, Apr. 11 2013 Journal of Virology.
26. Mesenchymal stem cell derived extracellular vesicles for the treatment of acute respiratory distress syndrome, Aswin Abraham, Stem Cells, 2020.
27. https://www.ncbi.nlm.nih.gov/pubmed/19131962
28. Isolation and purification of EVs in a Morpho butterfly wing-integrated micro cortex biochip, Shangying Han 2020, https://www.sciencedirect.com/science/article/pii/S0956566320300701.
29. Amanda Keener: “Exosomes make their Debut in Plant Research” The Scientist, Febraury 2019.
30. Haseeb Anwar Dad, Ting-Wei Gu, Ao-Qing Zhu, and Ling-Hua Peng, “Plant Exosome-like Nanovesicles: Emerging Therapeutics and Drug Delivery Nanoplatforms,” Molecular Therapy, January 2021.
31. Haiying Zhang, David Lyden: “Asymmetric -flow field-flow fractionation technology for exomere and small extracellular vesicle separation and characterization,” Nature protocols, https://doi.org/10.1038/s41596-019-0126-x, April 2019.
32. Zhuyuan Qin, Qingwen Xu, Haihong Hu, Lushan Yu, and Su Zeng. “Extracellular Vesicles in Renal Cell Carcinoma: Multifaceted Roles and Potential Applications Identified by Experimental and Computational Methods,” Frontiers in Oncology, May 2020.
33. Oguz Kaan Kirbas, “ Optimized Isolation of Extracellular Vesicles from Various Organic Sources using Aqueous Two-Phase System,” Scientific Reports Nature Research, 2019.
34. Carolina Paganini, Umberto Palmiero, Gabriella Pocsfalvi, Nicolas Touzet, Antonella Bongiovanni, Paolo Arosio, “Scalable Production and Isolation of Extracellular Vesicles: Available Sources and lessons from current Industrial Bioprocesses,” Biotechnology Journal, 2019.
35. James Inglese, Ronald L Johnson, Anton Simeonov, Menghang Xia, Wei Zheng, Christopher P Austin, & Douglas S Auld, “High Throughput screening assays for the identification of chemical probes,” Nature chemical Biology, 18 Jul. 2007.
36. Assay Development & Screening|National Center for Advancing Translational Sciences (nih. gov)
37. Katherine Crawford. “Protocol and Reagents for Pseudotyping Lentiviral Particles with SARS-CoV-Spike Protein for Neutralization Assays, Viruses, 2020.
38. Xin Huang. Xiaohui Lin, Jun Zeng. Lichao Wang, Peiyuan Yin, Lina Zhou, Chinxiu Hu, Weihong Yao, “A Computational Method of Defining Potential Biomarkers based on Differential Sub-Networks, Scientific Reports, October 2017.
39. J. Withrow, et al., “Extracellular Vesicles in the Pathogenesis of Rheumatoid Arthritis and Osteoarthritis,”Arthritis. Res. Ther., 2016; 18:286.

We claim:

1. A method of obtaining a subset of extracellular vesicles usable in the manufacture of drug-loaded extracellular vesicles configured to treat a disease or condition, the method comprising:
choosing the disease or condition for treatment with drug-loaded extracellular vesicles;
choosing a source of cells for naturally producing the extracellular vesicles by identifying the cells involved in a pathogenesis of the disease or the condition;
culturing, harvesting and isolating the extracellular vesicles from the cell source;
fractionating the characterized extracellular vesicles using a secondary assay method to obtain a subset of extracellular vesicles; and
characterizing and identifying the subset of extracellular vesicles prior to the manufacture of drug-loaded extracellular vesicles configured for treatment of the disease or the condition.

2. The method of claim 1, wherein the disease comprises a COVID-19 respiratory disease caused by a SARS-CoV-2 infection in a human patient.

3. The method of claim 1, wherein the condition is pain or inflammation.

4. The method of claim 1, wherein culturing, harvesting and isolating comprises at least one of a centrifugation method, a sucrose gradient method, a density gradient method, a filtration method, a precipitation method, a size-exclusion method, or an affinity method.

5. The method of claim 1, wherein fractionating comprises nanoparticle separation in an asymmetric flow field-flow fractionation (AF4) instrument.

6. The method of claim 1, wherein characterizing and identifying comprises at least one of atomic force microscopy (AFM), dynamic light scattering, flow cytometry, immunoblotting, immune-sorbent analysis, transmission electron microscopy (TEM), nanoparticle tracking analysis (NTA), scanning electron microscopy (SEM) or tunable resistive pulse-sensing.

7. A method of manufacturing drug-loaded extracellular vesicles configured for the treatment of a disease or a condition in a patient, the method comprising:
culturing cells capable of naturally producing extracellular vesicles;
isolating the naturally produced extracellular vesicles;
purifying the extracellular vesicles; and
incubating the purified extracellular vesicles in a buffer comprising the drug for a time sufficient to load the extracellular vesicles ex vivo with the drug to produce the drug-loaded extracellular vesicles.

8. The method of claim 7, wherein at least one of the cells or the naturally produced extracellular vesicles takes part in a pathogenesis of the disease or the condition.

9. The method of claim 7, wherein the cells comprise mesenchymal stem cells or monocyte derived myeloid cells.

10. The method of claim 7, wherein the disease comprises a COVID-19 respiratory disease caused by a SARS-CoV-2 infection in a human patient.

11. The method of claim 7, wherein the drug comprises at least one of casirivimab, imdevimab, or remdesivir.

12. The method of claim 7, wherein the condition comprises pain or inflammation.

13. The method of claim 7, wherein the drug comprises at least one of phytocannabinoid or an endocannabinoid.

14. A pharmaceutical composition configured to treat a disease or a condition, comprising:
extracellular vesicles; and
a drug encapsulated in the extracellular vesicles,
wherein the extracellular vesicles are naturally produced from cells involved in a pathogenesis of the disease or the condition, and
wherein the drug comprises at least one of an antibody, antibiotic, antiviral, antifungal, antitumor, a protein, a nucleic acid, an oligonucleotide, a chemotherapeutic, or a cannabinoid.

15. The composition of claim 14, wherein the disease comprises COVID-19 caused by a SARS-CoV-2 infection, cancer, mental illness, a neurodegenerative disease, an autoimmune disease, or epilepsy.

16. The composition of claim 14, wherein the condition comprises pain or inflammation.

17. The composition of claim 14, wherein the drug comprises at least one of casirivimab, imdevimab, or remdesivir.

18. The composition of claim 14, wherein the extracellular vesicles further comprise a biomolecule attached thereon.

19. The composition of claim 18, wherein the biomolecule is selected from the group consisting of nucleotides, nucleosides, oligonucleotides, DNA, RNA, hybridization probes, amino acids, polypeptides, proteins and fragments thereof, and antibodies and fragments thereof

20. The composition of claim 18, wherein the biomolecule comprises an ACE2 receptor antibody or a polypeptide capable of hybridizing to a portion of an ACE2 receptor.
